(19) United States
(12) Patent Application Publication
(10) Pub. No.: US 2008/0081826 A1
(43) Pub. Date: Apr. 3, 2008

(54) SYSTEM FOR EFFECTING WEIGHT LOSS

(76) Inventors: Damien P. Springuel, Glencoe, IL (US); Jayme R. Ford, Mount Prospect, IL (US); Richard Houghton, Eastchurch Sheerness Kent, ME (US); Margaret A. McCue, Chicago, IL (US); Kathryn McFarland, Lake Forest, IL (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/741,378

(22) Filed: Apr. 27, 2007

Related U.S. Application Data
(60) Provisional application No. 60/795,832, filed on Apr. 28, 2006.

(51) Int. Cl. A61K 31/135; A61K 31/4523; A61P 3/00

(52) U.S. Cl. 514/326; 514/650

(57) ABSTRACT

The present invention relates to a method for effecting or maintaining weight loss in an individual in need thereof. The method includes the administration of a weight loss agent in conjunction with a meal replacement formulation. The weight loss agent can be administered separately or co-formulated with the meal replacement agent. The method of the present invention exhibits a beneficial effect wherein the weight loss or weight loss maintenance observed with administration of the weight loss agent with the meal replacement formulation is greater than with the administration of either the weight loss agent or meal replacement formulation alone.

### CROSS REFERENCE TO RELATED APPLICATIONS

The present application claims the benefit of U.S. Provisional Application No. 60/795,8327 filed on Apr. 28, 2006, which is hereby incorporated in its entirety by reference.

### FIELD OF THE INVENTION

This invention relates to a system and methods for effecting weight loss and weight maintenance in an individual including a combination therapy including the co-administration of a weight loss agent and a meal replacement. The co-administration of the weight loss agent and the meal replacement formulation leads to greater weight loss than administration of either the agent or formulation alone. The weight loss agent and the meal replacement formulation can be taken or administered separately or in a coformulated mixture.

### BACKGROUND

It is estimated that approximately 97 million American adults are overweight or obese, a condition that substantially increases the risk of morbidity from diabetes, hypertension, stroke, heart disease, sleep apnea, osteoarthritis, respiratory problems, and certain cancers. Obesity is defined as a BMI (body mass index) over 30 kg/m², People with a BMI between 25 and 29.9 are considered overweight, but not obese Obesity is the number one nutritional problem in the United States. In the last decade alone, the percentage of adults aged 20 or older who are overweight or obese has skyrocketed to 64.5% (Flegal; JAMA 2002). As a leading cause of preventable death in the United States today, overweight and obesity represent a major public health challenge. The NIH-NHLBI Guidelines for the Treatment of Obesity continually stress the tremendous need for weight management initiatives in health care settings.

Clinical studies have suggested that moderate, sustained weight loss can reduce or eliminate surrogate markers of obesity-related disorders such as type 2 diabetes, hypertension and dyslipidemia. Unfortunately, long-term data show that most patients who lose weight regain their lost weight within 5 years, and that these disease-associated risk factors are re-established in patients with abnormal biomarkers, for example, increased fasting glucose, triglycerides or low HDL-cholesterol, at the beginning of weight loss.

There are myriad weight loss products on the market today that are sold as both over-the-counter products and as prescribed medications. The over-the-counter weight loss products include meal replacement formulations designed to be taken as a substitute for a full meal, with a low level of calories (e.g., <200 kcal), providing nutritious, palatable, satisfying and “quick” substitute meals (see, e.g., SLIMFAST®, OPTIFAST®, etc.). In addition, there are a number of prescription medications that are known to be effective weight loss agents, e.g., sibutramine (Hansen et al., Am. J. Clin. Nutr., 68(6): 1180-1186 (1998); Walsh et al., Int. J. Obes. Relat. Metab. Disord., 23: 1009-1015 (1999); James et al., Lancet, 356: 2119-2125 (2000); Apfelbaum et al., Am. J. Med., 106: 179-184 (1999), orlistat (Davidson, M. H. et al. (1999) JAMA 281:235-42), and phentermine (Douglas, A. et al. (1983) Int. J. Obes. 7:591-5), these agents have demonstrated a weight loss of about 5%-10% of body weight for drug compared to placebo.

In addition, there are a number of studies that indicate a lifestyle modification component, (e.g., exercise guidelines, consultation with physicians and dieticians) can enhance the effect of a weight loss program that includes either a meal replacement component or a weight loss agent component (Heymsfield et al., Intl. J. Obes., 27: 537-549 (2003); Wadden et al., Arch. Intern. Med., 161: 218-227 (2001), Foster et al., Am. J. Clin. Nutr., 82: 230S-235S (2005); Ditschuneit et al., Am. J. Clin. Nutr. 69: 198-204 (1999); Heshka et al., JAMA, 289: 1792-1798 (2003); Tsai et al., Ann. Intern. Med., 142: 56-66 (2005); Foster et al., Arch. Intern. Med., 161: 2133-2139 (2001); Flechtner-Mors et al., Obesity Research, 8(5): 399-402 (2000); Ashley et al., Arch. Intern. Med., 161: 1599-1604 (2001); Rothacker et al., Journal of the American Dietetic Association, 101(3): 345-347 (2001); Ahrens et al., Journal of the American Pharmacists Association, 43(5): 583-589 (2003).)

Also, it has been demonstrated that obese subjects with insulin sensitivity can also benefit from a weight loss program that includes a meal replacement component by improving glycemic control. Obese patients with insulin sensitivity placed on a weight loss program that included a meal replacement component demonstrated, in addition to weight loss, improvements, i.e., reductions, in levels of glucose, insulin, lipids, hemoglobin A1c, and hypertension. (Hensrud, D., Obesity Research, 9(4): 348S-353S (2001); Yip et al., Obesity Research, 9(4): 341S-347S (2001).)

Some preliminary work has been performed on the effect of weight loss when combining meal replacements with weight loss agents. However, although weight loss is enhanced, the results have been inconclusive as to whether it is attributable to the combination of the two components or whether it is due to either one of the components alone. (Aronne et al., Diabetes, 54: A437 (2005); Arrone et al., American Diabetes Association—65^{th} Scientific Sessions, (June 2005); Early et al., Endocrine Society's 87^{th} Annual Meeting (June 2005); Fernstrom et al., Amer. J. Clin. Nutr., 75: 375S (2002); Fernstrom et al., Obesity, 9:68S (2001); Redmon et al., Diabetes, 28: 1311-1315 (2005); Redmon et al., Diabetes Care, 26(9):2505-2511 (2003).

The present invention describes an effective method for weight loss and weight maintenance including a regimen including co-administration of a meal replacement component with a weight loss agent. It is demonstrated by this method that the combination of a meal replacement component with a weight loss agent improves weight loss and weight maintenance results over administration of either a meal replacement component alone or a weight loss agent alone.

### SUMMARY OF THE INVENTION

The present invention relates to a method for effecting or maintaining weight loss in an individual in need thereof. The method includes the administration of a weight loss agent in conjunction with a meal replacement formulation. The weight loss agent can be administered separately or co-formulated with the meal replacement agent. The method of the present invention exhibits a beneficial effect wherein the weight loss or weight loss maintenance observed with administration of the weight loss agent with the meal replacement formulation is greater than with the administration of either the weight loss agent or meal replacement formulation alone.

### DETAILED DESCRIPTION OF THE INVENTION

The present invention is directed to a method for effecting or maintaining weight loss in an individual in need of losing weight or maintaining weight loss including the coordinated administration to an individual of a weight loss agent in conjunction with a meal replacement formulation. The present invention contemplates a weight control system including a weight loss agent and a meal replacement formulation, adapted for co-administration such that use of the system is effective to cause greater weight loss than either component employed separately. The weight loss agent component and the meal replacement formulation component can be administered separately or together in a co-packaged or co-formulated form. A particular advantage of this method is the ease with which an individual following the method can comply with the steps of the method in that, in most cases, the individual needs a minimum of only one administration per day of the weight loss agent and a minimum of one administration pet day of the meal replacement formulation or one to two administrations per day of the co-formulation, which method can be performed at home without the assistance of a second party, e.g., physician, dietician, etc. However, the present method can include a counseling/monitoring component whereby an individual practicing the method of the present invention can meet one or more times with a physician, dietician, pharmacist and/or other health care provider for the purpose of receiving instructions on following the method and/or instructions on additional life style modifications for enhancing the weight loss effect of the method egg, regular exercise, and/or periodic visits with the physician or dietician to monitor the progress of weight loss or to provide motivation for continuing the weight loss program, Another advantage of the present method is that the remainder of the daily meals can be chosen from a variety of healthy, low calorie meals.

The present invention further contemplates a method of effecting or maintaining weight loss in an individual seeking to lose weight or maintain weight loss including co-administering to said individual of a weight loss agent in conjunction with a meal replacement formulation. Said co-administration of a weight loss agent in conjunction with a meal replacement formulation according to the invention leads to a greater weight loss or improved weight maintenance in said individual than the use of either the weight loss agent or the meal replacement formulation alone.

It is envisioned that the present method can optionally be performed in conjunction with a broader program designed to provide assistance to an individual in need of weight loss or weight maintenance and thereby maximize the beneficial effects of a diet that includes the method of the present invention. Thus, in a particular embodiment of the present invention, the method of effecting or maintaining weight loss is practiced in conjunction with a lifestyle modification program specifically tailored to the individual, which program can include, for example, a predetermined daily menu, fixed or regulated caloric intake, professional counseling or supervision, monitoring or recording of food intake, and/or programs of regular physical activity of exercise. Other suitable lifestyle modification programs include materials, lectures or sessions aimed at behavior modification, including educational literature, presentations, broadcasts, classes, group meetings, peer support, psychoanalysis, and the like.

A particular advantage of the present method for effecting or maintaining weight loss is the beneficial effect observed with the co-administration of the weight loss agent and the meal replacement formulation. In other words, an individual utilizing the system of the invention experiences a greater weight loss or better weight loss maintenance than by utilizing just the weight loss agent alone without the meal replacement formulation or just the meal replacement formulation alone without the weight loss agent. It is envisioned that the addition of a lifestyle modification program as described above also improves the weight loss or weight maintenance results experienced with the method of the present invention. Therefore, the method of the present invention represents a system for improving or maintaining weight loss wherein the weight loss or maintenance results experienced by co-administration of both components, i.e., the weight loss agent and the meal replacement formulation, are greater than with either component alone.

In another embodiment of the invention, the meal replacement formulation and the weight loss agent are provided as a kit that can include a number of individual meal replacement formulations and a number of individual doses of the weight loss agent to be taken in conjunction with or mixed directly (co-formulated) with the meal replacement formulation prior to ingestion. In this manner, the amount in the individual doses of the weight loss agent can be tailored to the specific prescription of the individual seeking weight loss.

In another embodiment of the invention, the kit can include behavior modification tools such as pedometer, meal plans, and a diary to record food intake or exercise.

In another embodiment of the invention, the invention provides a unique delivery system which provides an intensive weight loss program of a weight loss agent, a calorie controlled nutritional product and a behavior modification program, improving overall compliance, which results in improved weight loss and weight maintenance as compared to calorie restricted diet alone, meal replacement alone or a weight loss agent alone.

In another embodiment, the unique delivery system can include a pretreatment of for example, from 4 to 12 weeks with a calorie controlled nutritional product (meal replacement) followed by the use of a weight loss agent and a calorie controlled nutritional product (meal replacement) combined in a convenient kit to enhance the implementation of life style management changes, resulting in improved compliance to predefined diet/nutrition plans for individuals and improved weight loss and weight maintenance.

In another embodiment, the unique delivery system can include a pretreatment of, for example, from 4 to 12 weeks with a calorie controlled nutritional product and a behavior modification program followed by the use of a weight loss agent and a calorie controlled nutritional product combined with a specialized weight/behavioral management program of, for example, from 12 to 52 weeks. This is a convenient program, which enhances the implementation of life style changes and results in improved compliance to a predefined diet/nutrition plan for individuals, in turn resulting in improved weight loss and weight maintenance.

In another embodiment, the kit can include a number of meal replacement formulations and more than one type of weight loss agent, in particular, a first weight loss agent that is chemically distinct from a second weight loss agent. According to this embodiment, the first and second weight loss agents can be administered in an alternating cycle with each other wherein the first weight loss agent is administered with the meal replacement formulation for a set number of consecutive days, weeks, months, etch, then immediately following this set period, the second weight loss agent is administered with the meal replacement formulation, preferably but not necessarily for the same set number of days, weeks, months, etc. as the first weight loss agent. This alternating cycle can persist for the duration of the weight loss program. For example, according to this embodiment, in addition to the daily administration of the meal replacement formulation, the first weight loss agent can be taken in an alternating cycle with the second weight loss agent wherein the first weight loss agent is taken with the meal replacement formulation for a first 7-day period, then the second weight loss agent is taken with the meal replacement formulation for a second 7-day period immediately following the first 7-day period. This cycle can continue for the duration of the program. In another embodiment, this administration can include a 14-day cycle wherein the first weight loss agent is taken with the meal replacement formulation for a first 14-day period, then the second weight loss agent is taken with the meal replacement formulation for a second 14-day period immediately following the first 14-day period. This cycle can continue for the duration of the program. In yet another embodiment, the administration can include an alternating 28-day cycle wherein the first weight loss agent is administered with the meal replacement formulation for a first 28-day period and the second weight loss agent is administered with the meal replacement formulation for a second 28-day period immediately following the first 28-day period. This alternating cycle can continue for the duration of the weight loss program.

As used herein, the term “weight loss agent” is any chemical, compound, composition or drug that is effective to enhance satiety (fullness feeling), decrease appetite, or reduce fat absorption resulting in decreased caloric intake when administered to an individual. Many such weight loss agents are known and include, without limitation, amphetamine-type appetite suppressants, serotonin norepinephrine reuptake inhibitors, lipase inhibitors, selective cannabinoid receptor antagonists, selective 5-HT2C receptor agonists, β-3-adrenergic receptor agonists, neuropeptide-Y (PYY) analogues, thermogenesis mediators, incretin (GLP-1) and amylin analogues, DPPIV inhibitors, hormone ciliary neurotrophic factor (CNTF), cholecystokinin agonists, dopamine agonists, ghrelin antagonists, melanocortin MC4 agonists, selective serotonin reuptake inhibitors (SSRI), NPY-1 antagonists, histamine H3 receptor antagonists, tyrosine phosphatase inhibitors, antihyperglycemic agents, androgens, and the like. Numerous examples of such weight loss agents are discussed infra.

Weight loss agents can include satiety agents. As used herein, the term “satiety agent” is any chemical, compound, composition, or drug that is effective to produce a feeling of satiety (“fullness”) resulting in decreased caloric intake when administered to an individual. Many such satiety agents are known, including bulk satiety agents Bulk satiety agents swell in the stomach when taken with water, giving rise to a sense of satiety and thus reducing food intake. A typical formulation might include complex sugar, for example, as a source of energy to assist with enhanced fullness. Suitable bulk-forming agents include one or more of the following agents: methylcellulose, sterculia, wheat fiber/bran, isphagula husk, carboxymethylcellulose, hydroxyethylcellulose, hydroxypropylmethylcellulose, guar gum, xanthan gum, carrageenan gum, tragacanth, carob, agar, locust bean gum, sodium alginate, alginic acid or Matricor. A non-limiting example of such a bulk satiety agent is Celevac.

In a particularly preferred embodiment, the weight loss agent used in the present invention is sibutramine or a pharmaceutically acceptable salt of sibutramine. Sibutramine is a combined norepinephrine-serotonin reuptake inhibitor that is associated with increased satiety, i.e., fullness, aid a resulting reduction in food intake (see, Wadden et al., Arch. Intern. Med., 161: 218-227 (2001)).

In a preferred embodiment, the weight loss agent useful in the present invention is orlistat or a pharmaceutically acceptable salt of orlistat. Orlistat is a gastric and pancreatic lipase inhibitor that induces weight loss by blocking the absorption of about one third of the fat contained in a meal. (see, Torgerson et al., Diabetes Care, Vol. 7, No. 1, 155-161 (1994).)

In another preferred embodiment, the weight loss agent useful in the present invention is rimonabant or a pharmaceutical salt of rimonabant. Rimonabant is a CB1 cannabinoid receptor antagonist that is being developed as a weight loss drug. The use of other, alternative weight loss and satiety agents is also contemplated, as discussed infra.

As used herein, “meal replacement formulations” refers to any nutritional composition of fixed or measured caloric content, whether supplemented with vitamins, fiber additives, minerals, or other nutritional dietary ingredients, which are intended for consumption by an individual as a substitute for a regular meal. The meal replacement formulation can be in liquid, solid, or semi-solid form. Numerous meal replacement formulations are known and are discussed infra.

In a preferred embodiment, the meal replacement formulation used in the system and method of the present application can include any known commercially available over-the-counter weight loss products. For example, the meal replacement formulation can include such commercially available products as ENSURE®, etc. Other forms of meal replacement formulation envisioned by the present method include protein bars, powders, and solid crisp matrix, e.g., rice crisps. A more complete list of meal replacement formulations contemplated by the present invention is provided below.

In a preferred weight loss or weight loss maintenance method of the present invention, an overweight or obese individual in need of losing weight or maintaining weight loss can be administered a dose of sibutramine in conjunction with a meal replacement formulation. In one embodiment, the sibutramine and meal replacement formulation can be taken separately. In a particularly preferred embodiment, the sibutramine and meal replacement formulation can be pre-packaged as a co-formulation for the individual upon purchase or packaged in pre-measured amounts to be mixed together by the individual before ingestion. In this way, the method of the present invention can be completed in one step that is easy and convenient to perform and is therefore more likely to represent a weight loss program that the individual can adhere to. In a preferred embodiment, the present application includes a method for effecting or maintaining weight loss in an individual seeking to lose weight or maintain weight loss including co-administration to said individual of a) a weight loss agent, and b) a meal replacement formulation, wherein the co-administration to said individual of said weight loss agent (a) in combination with said meal replacement formulation (b) effects a greater weight loss in said individual than the administration of either agent (a) or (b) alone.

In another preferred embodiment of this method, the weight loss agent and the meal replacement formulation are administered in a co-formulation. In another preferred embodiment, the weight loss agent and the meal replacement formulation are administered in separate doses. In another embodiment, the meal replacement formulation can include, but is not limited to, GLUCERNA®, GLUCERNA WEIGHT LOSS SHAKE, GLUCERNA SR, GLUCERNA MEAL BARS, GLUCERNA SELECT, GLUCERNA SHAKE, GLUCERNA SNACK BARS, GLUCERNA SHAKE CONSUMERS, GLUCERNA POWDER PROMOTE, SLIMFAST, SLIMFAST OPTIMA, ENSURE, BOOST, OPTIFAST, MODIFAST, PROMOTE WITH FIBER, PROSURE SHAKE, ENRICH, ISOCAL, ALMASED, BIO NORM, MULTABEN, HUMANA, SLIMFAST ENERGIE RIEGEL, GERLINEA, GAYELORD HAUSER, GAYELORD MINCEUR, MILICAL, PROTICAL, YOPLAIT, DIET CENTURY, CANDIA, MATIN FONDANT, MATIN CROQUANT, MATIN MOELLEUX, WEIGHT CARE, COMPLAN, SHAPERS, BUILD-UP, ADVANTAGE, DUNN'S RIVER, NUTRASLIM, MICRO DIET, SLIMTOP, MEIJI, PERFECT PLUS, DIET PROGRAM, HERBALIFE THERMOJETICS, DIET SENGEN, DIET G8, MEDI-F, TERU MEAL, ACT CARE, ISOTENDER, PESOFORMA, PIATTORICCO, ENERVIT, NATURLAND, ZONE BAR, ZONE PERFECT BAR, MYOPLEX, BODY FOR LIFE DRINK, and BODY FOR LIFE POWDER.

In a more preferred embodiment, the meal replacement formulation of the present invention contains 180 to 300 kcal/serving, 35-55% carbohydrate (% total kcal), 15-25% protein (% total kcal) and 20-35% fat (% total kcal). This meal replacement contains a blend of corn maltodextrin, sugar alcohols, fructose, soy fiber and fructooligosaccarides (FOS) as carbohydrate. This meal replacement formulation can be in the form of a reconstituted powder, liquid or meal bar.

In yet another embodiment, the weight loss agent is sibutramine or a pharmaceutically-acceptable salt of sibutramine including N,N-dimethyl-1-[1-(4-chlorophenyl)cyclobutyl]-3-methylbutylamine or a pharmaceutically-acceptable salt thereof, for example the hydrochloride salt, for example its monohydrate salt.

In another embodiment, the sibutramine is N-{1-[-(4-chlorophenyl)cyclobutyl]-3-methylbutyl}-N-methylamine and 1-[1-(4-chlorophenyl)cyclobutyl]-3-methylbutylamine or pharmaceutically acceptable salts thereof including the hydrochloride salt, for example its monohydrate salt.

In another embodiment, the sibutramine can be, but is not limited to, sibutramine hydrochloride, sibutramine mesylate, sibutramine hydrochloride monohydrate, sibutramine mesylate hemihydrate, or sibutramine methanesulfonate hemihydrate. In yet another embodiment of the present invention, the weight loss agent is an enantiomer of sibutramine including enantiomers including, but not limited to:
(+)-N-[1-[(4-chlorophenyl)cyclobutyl]-3-methylbutyl}-N-methylamine;
(−)-N-{1-[1-(4-chlorophenyl)cyclobutyl-3-methylbutyl}-N-methylamine;
(+)-1-[1-(4-chlorophenyl)cyclobutyl]-3-methylbutylamine;
(−)-1-[1-(4-chlorophenyl)cyclobutyl]-3-methylbutylamine;
(+)-N-{1-[1-(4-chlorophenyl)cyclobutyl]-3-methyl butyl}-N—N-dimethylamine; or
(−)-N-{1-[1-(4-chlorophenyl)cyclobutyl]-3-methylbutyl}-N—N-dimethylamine.

In a preferred embodiment, the sibutramine is administered in a dose of 0.1 mg/day to 100 mg/day. In a more preferred embodiment, the sibutramine is administered in a dose of from about 1 mg/day to about 50 mg/day. In a more preferred embodiment, the sibutramine is administered in a dose of 10 mg/day. In a more preferred embodiment, the sibutramine is administered in a dose of 115 mg/day. In a more preferred embodiment, the sibutramine is administered in a dose of 20 mg/day. In a more preferred embodiment, the dose of sibutramine is administered in a dose of 25 mg/day or 30 mg/day. In another embodiment, the dose of sibutramine can be, but is not limited to, 11 mg/day, 2 mg/day, 3 mg/day, 4 mg/day, 5 mg/day, 6 mg/day, 7 mg/day, 8 mg/day, 9 mg/day, 10 mg/day, 11 mg/day, 12 mg/day, 13 mg/day, 14 mg/day, 15 mg/day, 16 mg/day, 177 mg/day, 15 mg/day, 19 mg/day, 20 mg/day, or 30 mg/day. In a more preferred embodiment, the dose of sibutramine can be, but is not limited to, 1.0 mg/day, 1.25 mg/day, 1.50 mg/day, 1.75 mg/day, 2.0 mg/day, 2.25 mg/day, 2.50 mg/day, 2.75 mg/day, 3.0 mg/day, 3.25 mg/day, 3.50 mg/day, 3.75 mg/day, 4.0 mg/day, 4.25 mg/day, 4.50 mg/day, 4.75 mg/day, 5.0 mg/day, 5.25 mg/day, 5.50 mg/day, 5.75 mg/day, 6.0 mg/day, 6.25 mg/day, 6.50 mg/day, 6.75 mg/day, 7.0 mg/day, 7.25 mg/day, 7.50 mg/day, 7.75 mg/day, 8.0 mg/day, 8.25 mg/day, 8.50 mg/day, 8.75 mg/day, 9.0 mg/day, 9.25 mg/day, 9.50 mg/day, 9.75 mg/day, 10.0 mg/day, 10.25 mg/day, 10.50 mg/day, 10.75 mg/day, 11.0 mg/day, 11.25 mg/day, 11.50 mg/day, 11.75 mg/day, 12.0 mg/day, 12.25 mg/day, 12.50 mg/day, 12.75 mg/day, 13.0 mg/day, 13.25 mg/day, 13.50 mg/day, 13.75 mg/day, 14.0 mg/day, 14.25 mg/day, 14.50 mg/day, 14.75 mg/day, 15.0 mg/day, 15.25 mg/day, 15.50 mg/day, 15.75 mg/day, 16.0 mg/day, 16.25 mg/day, 16.50 mg/day, 16.75 mg/day, 17.0 mg/day, 17.25 mg/day, 17.50 mg/day, 17.75 mg/day, 18.0 mg/day, 18.25 mg/day, 18.50 mg/day, 18.75 mg/day, 19.0 mg/day, 19.25 mg/day, 19.50 mg/day, 19.75 mg/day, 20.0 mg/day, or 30.0 mg/day.

In another embodiment of the method of the present invention, the meal replacement formulation can be, but is not limited to, a liquid, a bar, or a powder, and the weight loss agent is sibutramine.

In one embodiment, the meal replacement formulation is from 50 kcal to 100 kcal and the daily dose of sibutramine is from 0.1 mg to 100 mg. In another embodiment, the dose of sibutramine is 1 mg/day to 50 mg/day. In yet another embodiment, the dose of sibutramine is 10 mg/day. In yet another embodiment, the dose of sibutramine is 15 mg/day. In still another embodiment, the dose of sibutramine is 20 mg/day. In yet another embodiment, the dose of sibutramine is 25 mg/day. In still another embodiment, the dose of sibutramine can be, but is not limited to, 1 mg/day, 2 mg/day, 3 mg/day, 4 mg/day, 5 mg/day, 6 mg/day, 7 mg/day, 8 mg/day, 9 mg/day, 10 mg/day, 1 mg/day, 122 mg/day, 13 mg/day, 14 mg/day, 15 mg/day, 16 mg/day, 17 mg/day, 18 mg/day, 19 mg/day, 20 mg/day, or 30 mg/day.

In a more preferred embodiment, the dose of sibutramine can be, but is not limited to, 1.0 mg/day, 1.25 mg/day, 1.50 mg/day, 1.75 mg/day, 2.0 mg/day, 2.25 mg/day, 2.50 mg/day, 2.75 mg/day, 3.0 mg/day, 3.25 mg/day, 3.50 mg/day, 3.75 mg/day, 4.0 mg/day, 4.25 mg/day, 4.50 mg/day, 4.75 mg/day, 50 mg/day, 5.25 mg/day, 5.50 mg/day, 5.75 mg/day, 6.0 mg/day, 6.25 mg/day, 6.50 mg/day, 6.75 mg/day, 7.0 mg/day, 7.25 mg/day, 7.50 mg/day, 7.75 mg/day, 8.0 mg/day, 8.25 mg/day, 8.50 mg/day, 8.75 mg/day, 9.0 mg/day, 9.25 mg/day, 9.50 mg/day, 9.75 mg/day, 10.0 mg/day, 10.25 mg/day, 10.50 mg/day, 10.75 mg/day, 11.0 mg/day, 11.25 mg/day, 11.50 mg/day, 11.75 mg/day, 12.0 mg/day, 12.25 mg/day, 12.50 mg/day, 12.75 mg/day, 13.0 mg/day, 13-25 mg/day, 13.50 mg/day, 13.75 mg/day, 14.0 mg/day, 14.25 mg/day, 14.50 mg/day, 14.75 mg/day, 15.0 mg/day, 15.25 mg/day, 15.50 mg/day, 15.75 mg/day, 16.0 mg/day, 16.25 mg/day, 16.50 mg/day, 16.75 mg/day, 17.0 mg/day, 17.25 mg/day, 17.50 mg/day, 17.75 mg/day, 18.0 mg/day, 18.25 mg/day, 18.50 mg/day, 18.75 mg/day, 19.0 mg/day, 19.25 mg/day, 19.50 mg/day, 19.75 mg/day, 20.0 mg/day, or 30.0 mg/day.

In another embodiment of the method of the present invention the meal replacement formulation is from 150 kcal to 400 kcal and the daily dose of sibutramine is from 0.1 mg to 100 mg. In a preferred embodiment, the dose of sibutramine is 1 mg/day to 50 mg/day. In another embodiment, the dose of sibutramine is 10 mg/day. In yet another embodiment, the dose of sibutramine is 15 mg/day. In still another embodiment the dose of sibutramine is 20 mg/day. In still another embodiment, the dose of sibutramine is 25 mg/day. In yet another embodiment, the dose of sibutramine can be, but is not limited to, 1 mg/day, 2 mg/day, 3 mg/day, 4 mg/day, 5 mg/day, 6 mg/day, 7 mg/day, 5 mg/day, 9 mg/day, 10 mg/day, 11 mg/day, 12 mg/day, 13 mg/day, 14 mg/day, 15 mg/day, 16 mg/day, 17 mg/day, 18 mg/day, 19 mg/day, 20 mg/day, or 30 mg/day.

In a more preferred embodiment, the dose of sibutramine can be, but is not limited to, 1.0 mg/day, 1.25 mg/day, 1.50 mg/day, 1.75 mg/day, 2.0 mg/day, 2.25 mg/day, 2.50 mg/day, 2.75 mg/day, 3.0 mg/day, 3.25 mg/day, 3.50 mg/day, 3.75 mg/day, 4.0 mg/day, 4.25 mg/day, 4.50 mg/day, 4.75 mg/day, 5.0 mg/day, 5.25 mg/day, 5.50 mg/day, 5.75 mg/day, 6.0 mg/day, 6.25 mg/day, 6.50 mg/day, 6.75 mg/day, 7-0 mg/day, 7.25 mg/day, 7.50 mg/day, 7.75 mg/day, 8.0 mg/day, 8.25 mg/day, 8.50 mg/day, 8.75 mg/day, 9.0 mg/day, 9.25 mg/day, 9.50 mg/day, 9.75 mg/day, 10.0 mg/day, 10.25 mg/day, 10.50 mg/day, 10.75 mg/day, 11.0 mg/day, 11.25 mg/day, 11.50 mg/day, 11.75 mg/day, 12.0 mg/day, 12.25 mg/day, 12.50 mg/day, 12.75 mg/day, 13.0 mg/day, 13.25 mg/day, 13.50 mg/day, 13.75 mg/day, 14.0 mg/day, 14.25 mg/day, 14.50 mg/day, 14.75 mg/day, 15.0 mg/day, 15.25 mg/day, 15.50 mg/day, 15.75 mg/day, 16.0 mg/day, 16.25 mg/day, 16.50 mg/day, 16.75 mg/day, 17.0 mg/day, 17.25 mg/day, 17.50 mg/day, 17.75 mg/day, 18.0 mg/day, 18.25 mg/day, 18.50 mg/day, 18.75 mg/day, 19.0 mg/day, 19.25 mg/day, 19.50 mg/day, 19.75 mg/day, 20.0 mg/day, or 30.0 mg/day.

In a preferred embodiment the sibutramine is N,N-dimethyl-1-[1-(4-chlorophenyl)cyclobutyl]-3-methylbutylamine or a pharmaceutically acceptable salt thereof, for example is the hydrochloride salt, for example its monohydrate salt.

In yet another embodiment, the sibutramine is N-{1-[-(4-chlorophenyl)cyclobutyl]-3-methylbutyl}-N-methylamine and 1-[1-(4-chlorophenyl)cyclobutyl]-3-methylbutylamine or pharmaceutically acceptable salts thereof, for example the hydrochloride salt, for example its monohydrate salt.

In yet another embodiment, the sibutramine can be, but is not limited to, sibutramine hydrochloride, sibutramine mesylate, sibutramine hydrochloride monohydrate, sibutramine mesylate hemihydrate, or sibutramine methanesulfonate hemihydrate.

In still another embodiment, the satiety agent is an enantiomer of sibutramine, for example, but not limited to,
(+)-N-[1-[(4-chlorophenyl)cyclobutyl]-3-methylbutyl}-N-methylamine;
(−)-N-{1-[1-(4-chlorophenyl)cyclobutyl-3-methylbutyl}-N-methylamine;
(+)-1-[1-(4-chlorophenyl)cyclobutyl]-3-methlylbutylamine;
(−)-1-[1-(4-chlorophenyl)cyclobutyl]-3-methylbutylamine;
(+)-N-{1-[1-(4-chlorophenyl)cyclobutyl]-3-methylbutyl}-N—N-dimethylamine; or
(−)-N-{1-[1-(4-chlorophenyl)cyclobutyl]-3-methylbutyl}-N—N-dimethylamine.

In another embodiment of the method of the present invention, the weight loss agent is orlistat or a pharmaceutically acceptable salt of orlistat, for example (2S,3S,5S)-5-[(S)-2-formamido-4-methylvaleryloxy]-2-hexyl-3-hydroxyhexadecanoic acid lactone or N-formyl-L-leucine, ester with (3S,4S)-3-hexyl-4-[(2)-2-hydroxytridecyl]-2-oxetanone.

In another embodiment, the orlistat can be, but is not limited to, (−)-tetrahydrolipstatin or (−)-tetrahydrolipistatin. In another embodiment, the orlistat is administered in a dose of from 50 mg/day to 1440 mg/day, preferably the orlistat is administered in a dose of from 120 mg/day to 720 mg/day, more preferably the orlistat is administered in a dose of from 120 mg/day to 360 mg/day.

In another embodiment of the method of the present invention, the meal replacement formulation is can be, but is not limited to, a liquid, a bar, or a powder % and the weight loss agent is orlistat. In a preferred embodiment, the meal replacement formulation is from 50 kcal to 1000 kcal and the dose of orlistat is from 50 mg/day to 1440 mg/day, preferably from 120 mg/day to 720 mg/day, and more preferably from 120 mg/day to 360 mg/day.

In another embodiment, the orlistat is preferably (2S,3S,5S)-5-[(S)-2-formamido-4-methylvaleryloxy]-2-hexyl-3-hydroxyhexadecanoic acid lactone or N-formyl-L-leucine, ester with (3S,4S)-3-hexyl-4-[(2S)-2-hydroxytridecyl]-2-oxetanone.

In yet another embodiment, the orlistat includes, but is not limited to, (−)-tetrahydrolipstatin or (−)-tetrahydrolipistatin. In yet another embodiment, the meal replacement formulation is from 150 kcal to 400 kcal and the dose of orlistat is from 50 mg/day to 1440 mg/day, more preferably from 120 mg/day to 720 mg/day, and most preferably from 120 mg/day to 360 mg/day. Preferably, in this embodiment, the orlistat is (2S,3S,5S)-5-[(S)-2-formamido-4-methylvaleryloxy]-2-hexyl-3-hydroxyhexadecanoic acid lactone or N-formyl-L-leucine, ester with (3S,4S)-3-hexyl-4-[(2S)-2-hydroxytridecyl]-2-oxetanone. Also, in this embodiment, the orlistat can be, but is not limited to, (−)-tetrahydrolipstatin or (−)-tetrahydrolipistatin.

In yet another embodiment of the present invention, the weight loss agent is rimonabant or a pharmaceutically acceptable salt of rimonabant, preferably 1-H-pyrazole-3-carboxamide, 5-(4-chlorophenyl)-1-(2-,4-dichlorophenyl)-4-methyl-N-1-piperidinyl. In another embodiment, the pharmaceutically acceptable salt of rimonabant can be, but is not limited to, benzenesulfonate, hydrobromide, hydrochloride, citrate, ethanesulfonate, fumarate, gluconate, iodate, isothionate, maleate, methanesulfonate (mesylate), methylenebis-β-oxynaphthoate, nitrate, oxalate, pamoate, phosphate, salicylate, succinate, sulfate, tartrate, theophyllinacetate or p-toluenesulfonate, and preferably the salt of rimonabant is rimonabant hydrochloride.

In another embodiment, the dose of rimonabant is from 2 mg/day to 20 mg/day, more preferably from 5 mg/day to 1 mg/day, even more preferably from 7 mg/day to 10 mg/day and most preferably 10 mg/day.

In yet another embodiment the daily dose of rimonabant can be, but is not limited to, 2 mg/day, 5 mg/day, 10 mg/day, 15 mg/day, or 20 mg/day, more preferably the rimonabant can be, but is not limited to, 1 mg/day, 2 mg/day, 3 mg/day, 4 mg/day, 5 mg/day, 6 mg/day, 7 mg/day, 8 mg/day, 9 mg/day, 10 mg/day, 11 mg/day, 12 mg/day, 13 mg/day, 14 mg/day, 15 mg/day, 16 mg/day, 17 mg/day, 18 mg/day, 19 mg/day, or 20 mg/day, and most preferably, can be, but is not limited to, 2.0 mg/day, 2.25 mg/day, 2.50 mg/day, 2.75 mg/day, 3.0 mg/day, 3.25 mg/day, 3.50 mg/day, 3.75 mg/day, 4.0 mg/day, 4.25 mg/day, 4.50 mg/day, 4.75 mg/day, 5.0 mg/day, 5.25 mg/day, 550 mg/day, 5.75 mg/day, 6.0 mg/day, 6.25 mg/day, 6.50 mg/day, 6.75 mg/day, 7.0 mg/day, 7.25 mg/day, 7.50 mg/day, 7.75 mg/day, 8.0 mg/day, 8.25 mg/day, 8.50 mg/day, 8.75 mg/day, 9.0 mg/day, 9.25 mg/day, 9.50 mg/day, 9.75 mg/day, 10.0 mg/day, 10.25 mg/day, 10.50 mg/day, 10.75 mg/day, 11.0 mg/day, 11.25 mg/day, 11.50 mg/day, 11.75 mg/day, 12.0 mg/day, 12.25 mg/day, 12.50 mg/day, 12.75 mg/day, 13.0 mg/day, 13.25 mg/day, 13.50 mg/day, 13.75 mg/day, 14.0 mg/day, 14.25 mg/day, 14.50 mg/day, 14.75 mg/day, 15.0 mg/day, 15.25 mg/day, 15.50 mg/day, 15.75 mg/day, 16.0 mg/day, 16.25 mg/day, 16.50 mg/day, 16.75 mg/day, 17.0 mg/day, 17.25 mg/day, 17.50 mg/day, 17.75 mg/day, 18.0 mg/day, 18.25 mg/day, 18.50 mg/day, 18.75 mg/day, 19.0 mg/day, 19.25 mg/day, 19.50 mg/day, 19.75 mg/day, or 20.0 mg/day.

In another embodiment of the present invention, the meal replacement formulation can be, but is not limited to, a liquid, a bar, or a powder, and the weight loss agent is rimonabant, Preferably, the meal replacement formulation is from 50 kcal to 1000 kcal and the dose of rimonabant is from 2 mg/day to 20 mg/day, preferably from 5 mg/day to 15 mg/day, more preferably from 7 mg/day to 10 mg/day, and most preferably 10 mg/day.

In a more preferred embodiment, the meal replacement formulation of the present invention contains 180 to 300 kcal/serving, 35-55% carbohydrate (% total kcal), 15-25% protein (% total kcal) and 20-35% fat (% total kcal). This meal replacement contains a blend of corn maltodextrin, sugar alcohols, fructose, soy fiber and FOS as carbohydrate. This meal replacement formulation can be in the form of a reconstituted powder, liquid or meal bar.

In another preferred embodiment, the dose of rimonabant can be, but is not limited to, 2 mg, 5 mg, 10 mg, 15 mg, and 20 mg, preferably 1 mg/day, 2 mg/day, 3 mg/day, 4 mg/day, 5 mg/day, 6 mg/day, 7 mg/day, 8 mg/day, 9 mg/day, 10 mg/day, 11 mg/day, 12 mg/day, 13 mg/day, 14 mg/day, 15 mg/day, 16 mg/day, 17 mg/day, 18 mg/day, 19 mg/day, or 20 mg/day. and most preferably, the dose of rimonabant can be, but is not limited to, 2.0 mg/day, 2.25 mg/day, 2.50 mg/day, 2.75 mg/day, 3.0 mg/day, 3.25 mg/day, 3.50 mg/day, 3.75 mg/day, 4.0 mg/day, 4.25 mg/day, 4.50 mg/day, 4.75 mg/day, 5.0 mg/day, 5.25 mg/day, 5.50 mg/day, 5.75 mg/day, 6.0 mg/day, 6.25 mg/day, 6.50 mg/day, 6.75 mg/day, 7.0 mg/day, 7.25 mg/day, 7.50 mg/day, 7.75 mg/day, 8.0 mg/day, 8.25 mg/day, 8.50 mg/day, 8.75 mg/day, 9.0 mg/day, 9.25 mg/day, 9.50 mg/day, 9.75 mg/day, 10.0 mg/day, 10.25 mg/day, 10.50 mg/day, 10.75 mg/day, 11.0 mg/day, 11.25 mg/day, 11.50 mg/day, 11.75 mg/day, 12.0 mg/day, 12.25 mg/day, 12.50 mg/day, 12.75 mg/day, 13.0 mg/day, 13.25 mg/day, 13.50 mg/day, 13.75 mg/day, 14.0 mg/day, 14.25 mg/day, 14.50 mg/day, 14.75 mg/day, 15.0 mg/day, 15.25 mg/day, 15.50 mg/day, 15.75 mg/day, 16.0 mg/day, 16.25 mg/day, 16.50 mg/day, 16.75 mg/day, 17.0 mg/day, 17.25 mg/day, 17.50 mg/day, 17.75 mg/day, 18.0 mg/day, 18.25 mg/day, 18.50 mg/day, 18.75 mg/day, 19.0 mg/day, 19.25 mg/day, 19.50 mg/day, 19.75 mg/day, or 20.0 mg/day.

In yet another embodiment of the method of the present invention, the meal replacement formulation and said weight loss agent are co-formulated in a liquid. In yet another embodiment of the method of the present invention, the meal replacement formulation and weight loss agent are administered as a single dose after reconstitution of the meal replacement formulation and weight loss agent by the patient, practitioner or pharmacist at the time of dispensing or administration.

In still another embodiment of the method of present invention, the meal replacement formulation and said weight loss agent are co-formulated in a bar. In yet another embodiment of the method of the present invention, the meal replacement formulation and said satiety agent are co-formulated in a powder. In another embodiment, the present invention is directed to a system for effecting improved weight loss and weight loss maintenance in an individual seeking to lose weight or maintain weight loss including (a) a weight loss agent and (b) a meal replacement formulation, said system having increased effectiveness for causing weight loss than either component used alone. As used herein, the terms “system”, “weight loss system” or “weight control system” are used to designate a combination therapy whereby the components of the system are administered to an individual in a coordinated fashion with each other such that administration of the components manifests itself in a greater weight loss in the individual than administration of either component alone. According to this embodiment, the weight loss agent and said meal replacement formulation are administered in a co formulation. Alternatively, the weight loss agent and said meal replacement formulation are administered in separate doses. According to this embodiment, the meal replacement formulation can be, but is not limited to, GLUCERNA®, GLUCERNA WEIGHT LOSS SHAKE, GLUCERNA SR, GLUCERNA MEAL BARS, GLUCERNA SELECT, GLUCERNA SHAKE, GLUCERNA SNACK BARS, GLUCERNA SHAKE CONSUMERS, GLUCERNA POWDER PROMOTE, SLIMFAST, SLIMFAST OPTIMA, ENSURE, BOOST, OPTIFAST, MODIFAST, PROMOTE, PROMOTE WITH FIBER, PROSURE SHAKE, ENRICH, ISOCAL, ALMASED, BIO NORM, MULTABEN, HUMANA, GERLINEA, GAYELORD HAUSER, GAYELORD MINCEUR, MILICAL, PROTICAL, YOPLAIT, DIET CENTURY, CANDIA, MATIN FONDANT, MATIN CROQUANT, MATIN MOELLEUX, WEIGHT CARE, COMPLAN, SHAPERS, BUILD-UP, ADVANTAGE, DUNN'S RIVER, NUTRASLIM, MICRO DIET, SLIMTOP, MEIJI, PERFECT PLUS, DIET PROGRAM, HERBALIFE THERMOJETICS, DIET SENGEN, DIET G8, MEDI-F, TERU MEAL, ACT CARE, ISOTENDER, PESOFORMA, PIATTORICCO, ENERVIT, NATURLAND, ZONE BAR, ZONE PERFECT BAR, MYOPLEX, BODY FOR LIFE DRINK, OR BODY FOR LIFE POWDER.

In another embodiment of this system, the weight loss agent is sibutramine or a pharmaceutically-acceptable salt of sibutramine, for example, N,N-dimethyl-1-[1-(4-chlorophenyl)cyclobutyl]-3-methylbutylamine or a pharmaceutically acceptable salt thereof, preferably the hydrochloride salt, and most preferably the hydrochloride salt is its monohydrate salt.

In another embodiment of this system, the sibutramine is N-{1-[(4-chlorophenyl)cyclobutyl]-3-methylbutyl}-N-methylamine and 1-[1-(4-chlorophenyl)cyclobutyl]-3-methylbutylamine or pharmaceutically acceptable salts thereof preferably the hydrochloride salt and most preferably the hydrochloride salt is its monohydrate salt.

In yet another embodiment of this system, the sibutramine can be, but is not limited to, sibutramine hydrochloride, sibutramine mesylate, sibutramine hydrochloride monohydrate, sibutramine mesylate hemihydrate, or sibutramine methanesulfonate hemihydrate.

In still another embodiment of this system, the weight loss agent is an enantiomer of sibutramine for example, but is not limited to,
(+)-N-[1-[(4-chlorophenyl)cyclobutyl]-3-methylbutyl}-N-methylamine;
(−)-N-{1-[1-(4-chlorophenyl)cyclobutyl-3-methylbutyl}-N-methylamine;
(+)-1-[1-(4-chlorophenyl)cyclobutyl]-3-methylbutylamine;
(−)-1-[1-(4-chlorophenyl)cyclobutyl]-3-methylbutylamine;
(+)-N-{1-[1-(4-chlorophenyl)cyclobutyl]-3-methylbutyl}-N—N-dimethylamine; or
(−)-N-{1-[1-(4-chlorophenyl)cyclobutyl]-3-methylbutyl}-N—N-dimethylamine.

In a preferred embodiment of this system, the sibutramine is administered in a dose of from about 0.1 mg/day to about 100 mg/day, preferably from about 1 mg/day to about 50 mg/day, more preferably about 10 mg/day, even more preferably about 15 mg/day, more preferably about 20 mg/day and preferably about 25 mg/day.

In still another embodiment of this system, the dose of sibutramine includes, but is not limited to, 1 mg/day, 2 mg/day, 3 mg/day, 4 mg/day, 5 mg/day, 6 mg/day, 7 mg/day, 8 mg/day, 9 mg/day, 10 mg/day, 11 mg/day, 12 mg/day, 13 mg/day, 14 mg/day, 15 mg/day, 16 mg/day, 17 mg/day, 18 mg/day, 19 mg/day, or 20 mg/day.

Most preferably, according to this system, the dose of sibutramine is selected from the group including, but not limited to, 1.0 mg/day, 1.25 mg/day, 1.50 mg/day, 1.75 mg/day, 2.0 mg/day, 2.25 mg/day, 2.50 mg/day, 2.75 mg/day, 3.30 mg/day, 3.25 mg/day, 3.50 mg/day, 3.75 mg/day, 4.0 mg/day, 4.25 mg/day, 4.50 mg/day, 4.75 mg/day, 5.0 mg/day, 5.25 mg/day, 5.50 mg/day, 5.75 mg/day, 6.0 mg/day, 6.25 mg/day, 6.50 mg/day, 6.75 mg/day, 7.0 mg/day, 7.25 mg/day, 7.50 mg/day, 7.75 mg/day, 8.0 mg/day, 8.25 mg/day, 8.50 mg/day, 8.75 mg/day, 9.0 mg/day, 9.25 mg/day, 9.50 mg/day, 9.75 mg/day, 10.0 mg/day, 10.25 mg/day, 10.50 mg/day, 10.75 mg/day, 11.0 mg/day, 11.25 mg/day, 11.50 mg/day, 11.75 mg/day, 12.0 mg/day, 12.25 mg/day, 12.50 mg/day, 12.75 mg/day, 13.0 mg/day, 13.25 mg/day, 13.50 mg/day, 13.75 mg/day, 14.0 mg/day, 14.25 mg/day, 14.50 mg/day, 14.75 mg/day, 15.0 mg/day, 15.25 mg/day, 15.50 mg/day, 15.75 mg/day, 16.0 mg/day, 16.25 mg/day, 16.50 mg/day, 16.75 mg/day, 17.0 mg/day, 17.25 mg/day, 17.50 mg/day, 17.75 mg/day, 18.0 mg/day, 18.25 mg/day, 18.50 mg/day, 18.75 mg/day, 19.0 mg/day, 19.25 mg/day, 19.50 mg/day, 19.75 mg/day, 20.0 mg/day, or 30.0 mg/day.

In yet another embodiment of this system, the weight loss agent is orlistat or a pharmaceutically acceptable salt of orlistat, for example, (2S,3S,5S)-5-[(S)-2-formamido-4-methylvaleryloxy]-2-hexyl-3-hydroxyhexadecanoic acid lactone or N-formyl-L-leucine, ester with (3S,4S)-3-hexyl-4-[(2S)-2-hydroxytridecyl]-2-oxetanone.

In another embodiment of this system, the orlistat includes, but is not limited to, (−)-tetrahlydrolipstatin or (−)-tetrahydrolipistatin.

In yet another embodiment of this system, the orlistat is administered in a dose of from about 50 mg/day to about 1440 mg/day, preferably from about 120 mg/day to about 720 mg/day, and most preferably from about 120 mg/day to about 360 mg/day.

In another embodiment of this system, the weight loss agent is rimonabant or a pharmaceutically acceptable salt of rimonabant, for example, 1-H-pyrazole-3-carboxamide, 5-(4-chlorophenyl)-1-(2-,4-dichlorophenyl)-4-methyl-N-1-piperidinyl.

In a preferred embodiment, the pharmaceutically acceptable salt of rimonabant can be, but is not limited to, benzenesulfonate, hydrobromide, hydrochloride, citrate, ethanesulfonate, fumarate, gluconate, iodate, isothionate, maleate, methanesulfonate (mesylate), methylenebis-β-oxynaphthoate, nitrate, oxalate, pamoate, phosphate, salicylate, succinate, sulfate, tartrate, theophyllinacetate or p-toluenesulfonate.

In a preferred embodiment, the salt of rimonabant is rimonabant hydrochloride. In another embodiment of this system, the rimonabant is administered in a dose from 2 mg/day to 20 mg/day, more preferably from 5 mg/day to 15 mg/day, more preferably from 7 mg/day to 10 mg/day, and most preferably, 10 mg/day. In yet another embodiment, rimonabant can be, but is not limited to, 2 mg/day, 5 mg/day, 10 mg/day, 15 mg/day, and 20 mg/day, more preferably the rimonabant is selected from the group including, but not limited to, 1 mg/day, 2 mg/day, 3 mg/day, 4 mg/day, 5 mg/day, 6 mg/day, 7 mg/day, 8 mg/day, 9 mg/day, 10 mg/day, 11 mg/day, 12 mg/day, 13 mg/day, 14 mg/day, 15 mg/day, 16 mg/day, 17 mg/day, 18 mg/day, 19 mg/day, or 20 mg/day, and most preferably the rimonabant is, but not limited to, 2.0 mg/day, 2.25 mg/day, 2.50 mg/day, 2.75 mg/day, 3.0 mg/day, 3.25 mg/day, 3.50 mg/day, 3.75 mg/day, 4.0 mg/day, 4.25 mg/day, 4.50 mg/day, 4.75 mg/day, 5.0 mg/day, 5.25 mg/day, 5.50 mg/day, 5.75 mg/day, 6.0 mg/day, 6.25 mg/day, 6.50 mg/day, 6.75 mg/day, 7.0 mg/day, 7.25 mg/day, 7.50 mg/day, 7.75 mg/day, 8.0 mg/day, 8.25 mg/day, 8.50 mg/day, 8.75 mg/day, 9.0 mg/day, 9.25 mg/day, 9.50 mg/day, 975 mg/day, 10.0 mg/day, 10.25 mg/day, 10.50 mg/day, 10.75 mg/day, 11.0 mg/day, 11.25 mg/day, 11.50 mg/day, 11.75 mg/day, 12.0 mg/day, 12.25 mg/day, 12.50 mg/day, 12.75 mg/day, 13.0 mg/day, 13.25 mg/day, 13.50 mg/day, 13.75 mg/day, 14.0 mg/day, 14.25 mg/day, 14.50 mg/day, 14.75 mg/day, 15.0 mg/day, 15.25 mg/day, 15.50 mg/day, 15.75 mg/day, 16.0 mg/day, 16.25 mg/day, 16.50 mg/day, 16.75 mg/day, 17.0 mg/day, 17.25 mg/day, 17.50 mg/day, 17.75 mg/day, 18.0 mg/day, 18.25 mg/day, 18.50 mg/day, 18.75 mg/day, 19.0 mg/day, 19.25 mg/day, 19.50 mg/day, 19.75 mg/day, or 20.0 mg/day.

In still another embodiment of this system, the meal replacement formulation is selected from the group including, but not limited to, a liquid, a bar, and a solid. In another aspect, the present invention is directed to a weight control regimen kit including (a) a weight loss agent and (b) one or more meal replacement formulations, the amounts of (a) and (b) being adapted for use in combination to promote more effective weight loss or maintenance of weight loss than is achieved using either component (a) or (b) alone.

In a preferred embodiment of this kit, the weight loss agent and the meal replacement formulation are administered in a co-formulation. In another preferred embodiment of this kit, the weight loss agent and said meal replacement formulation are administered in separate doses.

In a preferred embodiment of this kit, the meal replacement formulation is selected from the group including, but not limited to, GLUCERNA®, GLUCERNA WEIGHT LOSS SHAKE, GLUCERNA SR, GLUCERNA MEAL BARS, GLUCERNA SELECT, GLUCERNA SHAKE, GLUCERNA SNACK BARS, GLUCERNA SHAKE CONSUMERS, GLUCERNA POWDER PROMOTE, SLIMFAST, SLIMFAST OPTIMA, ENSURE, BOOST, OPTIFAST, MODIFAST, PROMOTE, PROMOTE WITH FIBER, PROSURE SHAKE, ENRICH, ISOCAL, ALMASED, BIO NORM, MULTABEN, HUMANA, SLIMFAST ENERGIE RIEGEL, GERLINEA, GAYELORD HAUSER, GAYELORD MINCEUR, MILICAL, PROTICAL, YOPLAIT, DIET CENTURY, CANDIA, MATIN FONDANT, MATIN CROQUANT, MATIN MOELLEUX, WEIGHT CARE, COMPLAN, SHAPERS, BUIL-UP, ADVANTAGE, DUNN'S RIVER, NUTRASLIM, MICRO DIET, SLIMTOP, MEIJI, PERFECT PLUS, DIET PROGRAM, HERBALIFE THERMOJETICS, DIET SENGEN, DIET G8, MEDI-F, TERU MEAL, ACT CARE, ISOTENDER, PESOFORMA, PIATTORICCO, ENERVIT, NATURLAND, ZONE BAR, ZONE PERFECT BAR, MYOPLEX, BODY FOR LIFE DRINK, or BODY FOR LIFE POWDER.

In a more preferred embodiment, the meal replacement formulation of the present invention includes 180 to 300 kcal/serving, 35-55% carbohydrate (% total kcal), 15-25% protein (% total kcal) and 20-35% fat (% total kcal). This meal replacement includes a blend of corn maltodextrin, sugar alcohols, fructose, soy fiber and FOS as carbohydrate. This meal replacement formulation can be in the form of a reconstituted powder, liquid or meal bar.

Also, in a preferred embodiment of this kit, the weight loss agent is sibutramine or a pharmaceutically-acceptable salt of sibutramine, preferably N,N-dimethyl-1-[1-(4-chlorophenyl)cyclobutyl]-3-methylbutylamine or a pharmaceutically-acceptable salt thereof, for example the hydrochloride salt and most preferably, the hydrochloride salt is its monohydrate salt.

In another embodiment, the sibutramine is N-{1-[-(4-chlorophenyl)cyclobutyl]-3-methylbutyl}-N-methylamine and 1-[1-(4-chlorophenyl)cyclobutyl]-3-methylbutylamine or pharmaceutically acceptable salts thereof, for example, the hydrochloride salt, and preferably, the hydrochloride salt is its monohydrate salt.

In yet another embodiment of this kit, the sibutramine can be, but is not limited to, sibutramine hydrochloride, sibutramine mesylate, sibutramine hydrochloride monohydrate, sibutramine mesylate hemihydrate, or sibutramine methanesulfonate hemihydrate.

In still another embodiment, the weight loss agent is an enantiomer of sibutramine, preferably the enantiomer of sibutramine can be, but is not limited to,
(+)-N-[1-[(4-chlorophenyl)cyclobutyl]-3-methylbutyl}-N-methylamine;
(−)-N-{1-[1-(4-chlorophenyl)cyclobutyl-3-methylbutyl}-N-methylamine;
(+)-1-[1-(4-chlorophenyl)cyclobutyl]-3-methylbutylamine;
(−)-1-[1-(4-chlorophenyl)cyclobutyl]-3-methylbutylamine;
(+)-N-{1-[1-(4-chlorophenyl)cyclobutyl]-3-methylbutyl}-N—N-dimethylamine; or
(−)-N-{1-[1-(4-chlorophenyl)cyclobutyl]-3-methylbutyl}-N—N-dimethylamine.

In a preferred embodiment of this kit, the sibutramine is administered in a dose of from 0.1 mg/day to 100 mg/day, preferably from 1 mg/day to 50 mg/day.

In another preferred embodiment, the dose of sibutramine is 10 mg/day, preferably 15 mg/day, more preferably 20 mg/day and more preferably 25 mg/day or 30 mg/day.

In another embodiment, the dose of sibutramine can be, but is not limited to, 1 mg/day, 2 mg/day, 3 mg/day, 4 mg/day, 5 mg/day, 6 mg/day, 7 mg/day, 8 mg/day, 9 mg/day, 10 mg/day, 11 mg/day, 12 mg/day, 13 mg/day, 14 mg/day, 15 mg/day, 16 mg/day, 17 mg/day, 18 mg/day, 19 mg/day, 20 mg/day, or 30 mg/day.

Most preferably, the dose of sibutramine can be, but is not limited to, 1.0 mg/day, 1.25 mg/day, 1.50 mg/day, 1.75 mg/day, 2.0 mg/day, 2.25 mg/day, 2.50 mg/day, 2.75 mg/day, 3.0 mg/day, 3.25 mg/day, 3.50 mg/day, 3.75 mg/day, 4.0 mg/day, 4.25 mg/day, 4.50 mg/day, 4.75 mg/day, 5.0 mg/day, 5.25 mg/day, 5.50 mg/day, 0.75 mg/day, 6.0 mg/day, 6.25 mg/day, 6.50 mg/day, 6.75 mg/day, 7.0 mg/day, 7.25 mg/day, 7.50 mg/day, 7.75 mg/day, 8.0 mg/day, 8.25 mg/day, 8.50 mg/day, 8.75 mg/day, 9.0 mg/day, 9.25 mg/day, 9.50 mg/day, 9.75 mg/day, 10.0 mg/day, 10.25 mg/day, 10.50 mg/day, 10.75 mg/day, 11.0 mg/day, 11.25 mg/day, 11.50 mg/day, 11.75 mg/day, 12.0 mg/day, 12.25 mg/day, 12.50 mg/day, 12.75 mg/day, 13.0 mg/day, 13.25 mg/day, 13.50 mg/day, 13.75 mg/day, 14.0 mg/day, 14.25 mg/day, 14.50 mg/day, 14.75 mg/day, 15.0 mg/day, 15.25 mg/day, 15.50 mg/day, 15.75 mg/day, 16.0 mg/day, 16.25 mg/day, 16.50 mg/day, 16.75 mg/day, 17.0 mg/day, 17.25 mg/day, 17.50 mg/day, 17.75 mg/day, 18.0 mg/day, 18.25 mg/day, 18.50 mg/day, 18.75 mg/day, 19.0 mg/day, 19.25 mg/day, 19.50 mg/day, 19.75 mg/day, 20.0 mg/day, or 30.0 mg/day.

In another embodiment of this kit, the weight loss agent is orlistat or a pharmaceutically acceptable salt of orlistat, for example, (2S,3S,5S)-5-[(S)-2-formamido-4-methylvaleryloxy]-2-hexyl-3-hydroxyhexadecanoic acid lactone.

In another embodiment, the orlistat is N-formyl-L-leucine, ester with (3S,4S)-3-hexyl-4-[(2S)-2-hydroxytridecyl]-2-oxetanone, In yet another embodiment, the orlistat can be, but is not limited to, (−)-tetrahydrolipstatin or (−)-tetrahydrolipstatin.

In yet another embodiment of this kit, the orlistat is administered in a dose of from 50 mg/day to 1440 mg/day, preferably from 120 mg/day to 720 mg/day and most preferably from 120 mg/day to 360 mg/day.

In another embodiment of the kit of the present invention, the weight loss agent is rimonabant or a pharmaceutically acceptable salt of rimonabant, for example, 1-H-pyrazole-3-carboxamide, 5-(4-chlorophenyl)-1-(2-,4-dichlorophenyl)-4-methyl-N-1-piperidinyl.

In another embodiment, the pharmaceutically acceptable salt of rimonabant is selected from the group including, but limited to, benzenesulfonate, hydrobromide, hydrochloride, citrate, ethanesulfonate, fumarate, gluconate, iodate, isothionate, maleate, methanesulfonate (mesylate), methylenebis-β-oxynaphthoate, nitrate, oxalate, pamoate, phosphate, salicylate, succinate, sulfate, tartrate, theophyllinacetate and p-toluenesulfonate.

In a more preferred embodiment, the salt of rimonabant is rimonabant hydrochloride, In another embodiment of this kit, the rimonabant is administered in a dose from 2 mg/day to 20 mg/day, preferably from 5 mg/day to 15 mg/day, more preferably from 7 mg/day to 10 mg/day, and most preferably 10 mg/day.

In still another embodiment, the daily dose of rimonabant can be, but is not limited to, 2 mg, 5 mg, 10 mg, 15 mg, and 20 mg. Preferably, the dose of rimonabant is selected from the group including, but not limited to, 1 mg/day, 2 mg/day, 3 mg/day, 4 mg/day, 5 mg/day, 6 mg/day, 7 mg/day, 8 mg/day, 9 mg/day, 10 mg/day, 11 mg/day, 12 mg/day, 13 mg/day, 14 mg/day, 15 mg/day, 16 mg/day, 17 mg/day, 18 mg/day, 19 mg/day, or 20 mg/day. Most preferably, the dose of rimonabant can be, but is not limited to, 2.0 mg/day, 2.25 mg/day, 2.50 mg/day, 2.75 mg/day, 3.0 mg/day, 3.25 mg/day, 3.50 mg/day, 3.75 mg/day, 4.0 mg/day, 4.25 mg/day, 4.50 mg/day, 4.75 mg/day, 5.0 mg/day, 5.25 mg/day, 5.50 mg/day, 5.75 mg/day, 6.0 mg/day, 6.25 mg/day, 6.50 mg/day, 6.75 mg/day, 7.0 mg/day, 7.25 mg/day, 7.50 mg/day, 7.75 mg/day, 8.0 mg/day, 8.25 mg/day, 8.50 mg/day, 8.75 mg/day, 9.0 mg/day, 9.25 mg/day, 9.50 mg/day, 9.75 mg/day, 10.0 mg/day, 10.25 mg/day, 10.50 mg/day, 10.75 mg/day, 11.0 mg/day, 11.25 mg/day, 11.50 mg/day, 11.75 mg/day, 12.0 mg/day, 12.25 mg/day, 12.50 mg/day, 12.75 mg/day, 13.0 mg/day, 13.25 mg/day, 13.50 mg/day, 13.75 mg/day, 14.0 mg/day, 14.25 mg/day, 14.50 mg/day, 14.75 mg/day, 15.0 mg/day, 15.25 mg/day, 15.50 mg/day, 15.75 mg/day, 16.0 mg/day, 16.25 mg/day, 16.50 mg/day, 16.75 mg/day, 17.0 mg/day, 17.25 mg/day, 17.50 mg/day, 17.75 mg/day, 18.0 mg/day, 18.25 mg/day, 18.50 mg/day, 18.75 mg/day, 19.0 mg/day, 19.25 mg/day, 19.50 mg/day, 19.75 mg/day, or 20.0 mg/day.

In another embodiment of this kit, the meal replacement formulation is selected from the group including, but not limited to, a liquid, a bar, and a powder.

In another embodiment of the kit of the present invention, the kit further includes lifestyle modification tools.

In another embodiment of the kit of the present invention, the weight loss agents are sibutramine and rimonabant.

In a preferred embodiment, the dose of sibutramine is from 5 mg to 30 mg of sibutramine hydrochloride monohydrate or the mole equivalent amount of a sibutramine compound and the dose of rimonabant is from 2 mg to 20 mg of rimonabant hydrochloride or the mole equivalent amount of a rimonabant compound.

In a more preferred embodiment, the dose of sibutramine is from 5 mg to 19 mg of sibutramine hydrochloride monohydrate or the mole equivalent amount of a sibutramine compound and the dose of rimonabant is 3 mg to 20 mg of rimonabant hydrochloride or the mole equivalent amount of a rimonabant compound.

In an even more preferred embodiment, the dose of sibutramine is from 6 mg to 18 mg of sibutramine hydrochloride monohydrate or the mole equivalent amount of a sibutramine compound and the dose of rimonabant is 4 mg to 20 mg of rimonabant hydrochloride or the mole equivalent amount of a rimonabant compound.

In a still more preferred embodiment, the dose of sibutramine is from 7 mg to 17 mg of sibutramine hydrochloride monohydrate or the mole equivalent amount of a sibutramine compound and the dose of rimonabant is 5 mg to 20 mg of rimonabant hydrochloride or the mole equivalent amount of a rimonabant compound.

In an even more preferred embodiment, the dose of sibutramine is from 8 mg to 16 mg of sibutramine hydrochloride monohydrate or the mole equivalent amount of a sibutramine compound and the dose of rimonabant is from 6 mg to 20 mg of rimonabant hydrochloride or the mole equivalent amount of a rimonabant compound.

In a more preferred embodiment, the dose of sibutramine is 9 mg to 15 mg of sibutramine hydrochloride monohydrate or the mole equivalent amount of a sibutramine compound and the dose of rimonabant is 7 mg to 20 mg of rimonabant hydrochloride or the mole equivalent amount of a rimonabant compound.

In a more preferred embodiment, the dose of sibutramine is 10 mg to 15 mg of sibutramine hydrochloride monohydrate or the mole equivalent amount of a sibutramine compound and the dose of rimonabant is 10 mg to 20 mg of rimonabant hydrochloride or the mole equivalent amount of a rimonabant compound.

In a more preferred embodiment, the dose of sibutramine is 10 mg of sibutramine hydrochloride monohydrate or the mole equivalent amount of a sibutramine compound and the dose of rimonabant is 5 mg of rimonabant hydrochloride or the mole equivalent amount of a rimonabant compound.

In another preferred embodiment, the dose of sibutramine is 15 mg of sibutramine hydrochloride monohydrate or the mole equivalent amount of a sibutramine compound and the dose of rimonabant is 5 mg of rimonabant hydrochloride or the mole equivalent amount of a rimonabant compound.

In a more preferred embodiment, the dose of sibutramine is 10 mg of sibutramine hydrochloride monohydrate or the mole equivalent amount of a sibutramine compound and the dose of rimonabant is 10 mg of rimonabant hydrochloride or the mole equivalent amount of a rimonabant compound.

In another preferred embodiment, the dose of sibutramine is 10 mg of sibutramine hydrochloride monohydrate or the mole equivalent amount of a sibutramine compound and the dose of rimonabant is 15 mg of rimonabant hydrochloride or the mole equivalent amount of a rimonabant compound.

In yet another preferred embodiment, the dose of sibutramine is 15 mg of sibutramine hydrochloride monohydrate or the mole equivalent amount of a sibutramine compound and the dose of rimonabant is 10 mg of rimonabant hydrochloride or the mole equivalent amount of a rimonabant compound.

In yet another preferred embodiment, the dose of sibutramine is 15 mg of sibutramine hydrochloride monohydrate or the mole equivalent amount of a sibutramine compound and the dose of rimonabant is 15 mg of rimonabant hydrochloride or the mole equivalent amount of a rimonabant compound.

In another preferred embodiment, the dose of sibutramine is 15 mg of sibutramine hydrochloride monohydrate or the mole equivalent amount of a sibutramine compound and the dose of rimonabant is 20 mg of rimonabant hydrochloride or the mole equivalent amount of a rimonabant compound.

In another embodiment, the meal replacement formulation and the weight loss agents can be provided in a 7-day kit including 7 doses/servings of sibutramine and meal replacement formulations and instructions for the administration of said weight loss agents and meal replacement formulations.

In another embodiment, the meal replacement formulation and the weight loss agents can be provided in a 14-day kit including 14 doses/servings of sibutramine and meal replacement formulations and instructions for the administration of said weight loss agents and meal replacement formulations.

In another embodiment, the meal replacement formulation and the weight loss agents can be provided in a 28-day kit including 28 doses/servings of sibutramine and meal replacement formulations and instructions for the administration of said weight loss agents and meal replacement formulations.

In one embodiment of this package, the weight loss agents include, but are not limited to, sibutramine and rimonabant administered in doses as described above. In a preferred embodiment of this package, the instructions include administration of sibutramine for a 14-day period followed by administration of rimonabant for a 14-day period.

In another embodiment of this package, the instructions include administration of sibutramine for a 28-day period followed by administration of rimonabant for a 28-day period. Further details and embodiments of the invention are disclosed below.

Table 2 is a table presenting data groups derived from parallel studies showing the superior effects of using a system for weight loss in accordance with the invention when compared to regimens utilizing either a weight loss agent alone or a meal replacement formulation alone. All subjects received moderate or intensive lifestyle management training, data Group 1 received this lifestyle management alone, data Group 2 received meal replacement therapy in addition, data Groups 3-10 received varying regimens of sibutramine therapy alone, data Groups 11 and 12 received the weight control system of the present invention.

As described above and below, the present invention is directed to a method for effecting weight loss or maintenance of weight loss in an individual in need of losing weight or maintaining weight loss including administration to an individual in need of losing weight a weight control system for controlled weight loss including a weight loss agent administered in conjunction with a meal replacement formulation. The present invention demonstrates that administration of the weight loss agent in conjunction with the meal replacement formulation effects a greater weight loss or maintenance of weight loss in an individual than the administration of either the weight loss agent or meal replacement formulation alone. As used herein, the reference to the weight control system effecting a greater weight loss or maintenance of weight loss than administration of either the weight loss agent alone or the meal replacement formulation alone, indicates that the total weight lost or total weight loss maintained by an individual practicing the method described herein, i.e., administering a weight loss agent in conjunction with a meal replacement formulation, is greater than the total weight loss or total weight loss maintained, an individual can experience if administered the weight loss agent in a program that does not include the meal replacement formulation and vice versa.

According to the present method, the weight loss agent and the meal replacement formulation can be administered separately or in a single co-formulation wherein a single dose of the weight loss agent is mixed with or dissolved in a single serving of the meal replacement formulation for administration (e.g., ingestion) together. In one embodiment, the weight loss agent can be a satiety agent co-formulated with the meal replacement formulation.

Suitable weight loss agents include any of the known chemical or pharmaceutical compounds or drugs that induce satiety (“fullness”), decrease caloric intake, or reduce fat absorption, thereby assisting in curbing food intake and leading to reduction in weight. Weight loss agents achieve their effects by various known physiological mechanisms, for example, by acting as adrenergic agonists, by acting as particular receptor inhibitors, or by increasing or decreasing the expression or activity of a satiety-regulating protein or hormone in the body. For example, weight loss agents suitable for use in the present invention include, but are not limited to, receptor antagonists, for example, rimonabant; lipase inhibitors, for example, orlistat; or serotonin norepinephrine reuptake inhibitors, for example, sibutramine; as well as other types of compounds discussed herein. The weight loss agent can be either a prescription medication requiring dispensation at the order of a physician or can dispensed as an over-the-counter (OTC) product not requiring a prescription.

Suitable meal replacement formulations can include any known nutritional dietary supplements that are formulated to provide a nutritious substitute for a full meal and which has a measured caloric content, for example, a low-calorie, fixed calorie, or high calorie formulation, depending on the regimen desired for the individual consuming it. Many meal replacement formulations are known and commercially available, usually as over-the-counter products advertised as weight loss aids. Such products are conventionally sold either in the form of a liquid, e.g., SLIMFAST®, or in solid form, for example as a “candy bar”, “protein bar”, in a solid crisp matrix, or in powder form for dissolution or dispersion in a liquid (e.g., water, milk, fruit juice) prior to ingestion. As used herein, the term “solid crisp matrix”, refers to light, crispy food products having a low bulk density character similar to rice crisps, corn crisps, or similar, other well known carbohydrate-containing materials.

Meal replacement formulations can further include additional additives such as, for example, proteins, carbohydrates, fiber, lipids, fat, oligosaccharides, vitamins, minerals, flavorings, food coloring, etch, as well as bulking or dispersing aids to enhance texture (e.g., “mouth feel”) or dispersibility in liquids.

The “solid crisp matrix” can also take the form of free flowing crisp particulates, bound aggregates of such particulates, and/or solid bar-like matrices, provided that all such particulates, aggregates, or matrices also have the requisite bulk density character Ideally, if the meal replacement formulation is administered as a solid crisp matrix, then, in addition to the weight loss agent, the crisp matrix can additionally include from about 10% to about 50% by weight of a soluble viscous fiber, from about 10% to about 89% by weight of a carbohydrate other than, and in addition to, the soluble viscous fiber, and from about 1% to about 50% by weight of protein. Additionally, the solid crisp matrix can be prepared in most any dietary product forms of any size or configuration, e.g., rounded or cylindrical, circular, or wafer-like, rectangular or in a conventional bar form, or random or other defined shapes. These product forms also include small bite-size solids, including those that are packaged as a plurality of bites within a single container or package. The solid crisp matrix can also be packaged as free flowing food particles, e.g., breakfast cereal, in an appropriate box or other package.

In addition, the solid crisp matrix can be partially or completely coated with any suitable low calorie coating material, including yogurt, chocolate, or other confectionary or otherwise flavored material.

In another embodiment of the method of the present invention the weight loss agent can be administered with a meal replacement formulation that includes a two-component carbohydrate mixture as described, for example in U.S. Pat. No. 6,774,111, incorporated herein by reference. The two-component carbohydrate mixture includes a source of fructose in combination with at least one readily digestible glucose polymer. As used herein, the term “digestible glucose polymer” refers to hydrolyzed starches and glucose oligomers, which are rapidly digested. The use of the fructose in the two component carbohydrate meal replacement formulation significantly decreases the glycemic response which is particularly beneficial if the meal replacement formulation, in combination with the weight loss agent, is administered to a diabetic patient in need of weight loss, in particular, a patient with type II diabetes. Further, this two-component carbohydrate mixture meal replacement formulation allows for easy incorporation into any of the liquid, powder, bar, or solid matrix examples described above. This two component carbohydrate mixture meal replacement formulation contains 180 to 300 kcal/serving, 35-55% carbohydrate (% total kcal), 15-25% protein (% total kcal) and 20-35% fat (% total kcal). This meal replacement contains a blend of corn maltodextrin, sugar alcohols, fructose, soy fiber and FOS as carbohydrate and it provides increased satiety over other meal replacements by providing a unique blend of slowly digestible carbohydrates which results in delayed gastric emptying and delayed absorption, leading to enhanced satiety, decreased postprandial glucose and insulin states, decreased insulin secretion and increased insulin sensitivity and enhanced feeling of fullness. The method of this embodiment of the present invention enhances satiety by two distinct mechanisms of action, that is, it provides an increased feeling of fullness and satiety at each meal and throughout the day thereby increasing the likelihood of the patient complying with a weight loss program. When the weight loss agent is also a satiety agent, for example sibutramine, the method of the invention enhances satiety by two mechanisms that is, the two component carbohydrate mixture meal replacement provides a feeling of fullness and sibutramine provides centrally mediated satiety enhancement resulting in a reduction of food intake at each meal. In addition sibutramine and the two-component carbohydrate mixture meal replacement can improve lean body mass, lipid parameters, specifically triglycerides and HDL levels, blood sugar levels, glycemic response and blood cholesterol levels.

In one embodiment, the carbohydrate component of the meal replacement formulation includes fructose and at least one digestible glucose polymer. In a preferred embodiment, the fructose component represents about 5-50 wt/wt % and the digestible glucose polymer represents about 50-95 wt/wt %, more preferably, the fructose component represents about 5-30 wt/wt % and the digestible glucose polymer represents about 70-95 wt/wt %, and most preferably the fructose component represents about 10-25 wt/wt % and the digestible glucose polymer represents about 75-90 wt/wt % of the carbohydrate component of the meal replacement formulation on a dry matter basis.

As an added advantage, by following the method of the present invention, the remainder of the daily meals can be chosen from a variety of palatable, low calorie courses either preselected as part of a program in conjunction with the weight loss method of the present invention, or chosen independently by the individual. Preferably, the program can include a regimen of physical exercise/life style modification to maximize the beneficial effects of practicing the method of the present invention.

Another particular advantage of the present method is that better weight loss or weight loss maintenance results are achieved by administration of the weight loss agent combined with the meal replacement formulation over administration of either component alone. Secondly, there exists a beneficial effect with the present weight loss or weight loss maintenance method in that it can be expected that an individual administered the weight loss agent either separately or co-formulated with the meal replacement formulation loses more overall weight or maintain more overall weight loss than a comparable individual administered the weight loss agent alone without the meal replacement formulation or an individual administered the meal replacement formulation alone without the weight loss agent. Another advantage of the present method is that lower % doses of the weight loss agent can be prescribed when administered in conjunction with or co-formulated with the meal replacement formulation. It should be understood that the present method contemplates that when the weight loss agent is administered separately from the meal replacement formulation, i.e., the weight loss agent is not co-formulated with the meal replacement formulation, the weight loss agent can be ingested in a timely manner with the meal replacement formulation, i.e., taken either before or following ingestion of the meal replacement formulation or vice versa. In another example, the weight loss agent can be taken first thing in the morning and the meal replacement formulation taken later in the day. One of the advantages of the invention is that the combination of a weight loss agent and meal replacement results in enhanced satiety over the use of the weight loss agent or meal replacement alone. As such, the patient feels fuller and is more likely to comply with the weight loss or weight loss maintenance regimen. Another advantage of the invention is its ease of use for patients. Providing a meal replacement as part of a weight loss or weight loss maintenance program relieves the patient of the burden of calculating the desired caloric intake for a meal. The invention is also easy for patients to use because it provides the weight loss agent and the meal replacement together. These advantages increase the likelihood of the patient complying with the weight loss or weight loss maintenance program. A further advantage of the invention is that it provides a structured regimen to the patient. The patient is provided with the weight loss agent and the meal replacement together, thus allowing the patient to substitute a meal replacement for a meal, thus making it easier for the patient to comply with the weight loss or weight loss maintenance regimen. Another advantage of the invention is that it can be used as part of a life style management regimen wherein use of a meal replacement is part of the regimen.

In a preferred embodiment, the meal replacement formulation of the present invention can be any number of known commercially available (over-the-counter) or prescription only meal replacement weight loss formulations. The meal replacement formulation can be in the form of a liquid, e.g., in the form of a “milkshake”, or solid, for example, a “candy bar”, “protein bar”, or other solid matrix, or in powder form. Examples of commercially available meal replacement formulations suitable for use in the present invention include, but are not limited to:

GLUCERNA ®, GLUCERNA WEIGHT LOSS SHAKE, GLUCERNA
SR, GLUCERNA MEAL BARS, GLUCERNA SELECT, GLUCERNA
SHAKE, GLUCERNA SNACK BARS, GLUCERNA SHAKE CONSU-
MERS, GLUCERNA POWDER PROMOTE, SLIMFAST, SLIMFAST
OPTIMA, ENSURE, BOOST, OPTIFAST, MODIFAST, ROMOTE,
PROMOTE WITH FIBER, PROSURE SHAKE, ENRICH, ISOCAL,
ALMASED, BIO NORM, MULTABEN, HUMANA, GERLINEA,
GAYELORD HAUSER, GAYELORD MINCEUR, MILICAL, PROTI-
CAL, YOPLAIT, DIET CENTURY, CANDIA, MATIN FONDANT,
MATIN CROQUANT, MATIN MOELLEUX, WEIGHT CARE, COM-
PLAN, SHAPERS, BUILD-UP, ADVANTAGE, DUNN'S RIVER,
NUTRASLIM, MICRO DIET, SLIMTOP, MEIJI, PERFECT
PLUS, DIET PROGAM, HERBALIFE THERMOJETICS, DIET
SENGEN, DIET G8, MEDI-F, TERU MEAL, ACT CARE, ISO-
TENDER, PESOFORMA, PIATTORICCO, ENERVIT, NATUR-
LAND, ZONE BAR, ZONE PERFECT BAR, MYOPLEX, BODY
FOR LIFE DRTNK, BODY FOR LIFE POWDER.

The meal replacement formulations listed above are particularly advantageous in that all provide a highly nutritious, palatable, and satisfying source of vitamins and minerals as well as carbohydrates and proteins and are low in fat. Most importantly, the meal replacement formulations are designed to provide a fixed or measured level of calories, so that attention to the number of calories per day being consumed by individuals using such products is easily done When used in conjunction with a weight loss agent in accordance with the present invention, weight loss achieved by the individual taking both components is unexpectedly improved in comparison to individuals taking either product alone.

If the weight loss agent of the present invention is administered separately from the meal replacement formulation, i.e., the weight loss agent is not co-formulated with the meal replacement but is taken in conjunction with the meal replacement formulation, the weight loss agent can be formulated and administered in an oral dosage form, for example, oral solution, elixir, emulsion, magna, gel, dry blend, sprinkle, tablets, fast dissolving tablets capsules, granules, syrups, aqueous or oil suspension, or unit of use container by methods well known in the art.

For example, tablets with the weight loss agent can be prepared from a mixture of the weight loss agent with fillers, for example calcium phosphate; disintegrating agents, for example maize starch; lubricating agents, for example magnesium stearate; binders, for example microcrystalline cellulose or polyvinylpyrrolidone and other optional ingredients known in the art to assist in the formation of tablets. In addition, the tablets can include an enteric coating such as, for example, hydroxypropylmethylcellulose phthalate. The term “enteric coating” covers any substance that facilitates rapid transport through the stomach to allow for dissolution in the intestine. The tablets can also be formulated in a manner known to those skilled in the art to give a sustained release of the weight loss agent of the present invention.

Also, the weight loss agent of the present invention, if administered separately from the meal replacement formulation, can be formulated and administered as hard or soft gelatin capsules containing the weight loss agent and prepared by known methods and, if desired, provided with enteric or other functional coatings in a known manner. If desired, the contents of the capsule can be formulated using known methods so as to give sustained or timed release of the weight loss agent.

Other dosage forms for separate oral administration of the weight loss agent of the present invention include aqueous suspensions containing the weight loss agent in an aqueous medium, preferably in the presence of a non-toxic suspending agent such as sodium carboxymethyl cellulose, and oily suspensions containing the weight loss agent in a suitable vegetable oil, for example arachis oil.

In another embodiment of the present invention, the weight loss agent can be formulated into granules with or without additional excipients. The granules can be ingested directly or added, i.e., dissolved in, or mixed with the meal replacement formulation before ingestion. The granules can contain disintegrants, eg. an effervescent couple formed from an acid and a carbonate or bicarbonate salt to facilitate dispersion in a liquid medium. By way of example, if the weight loss agent is administered by capsule, the capsule can include approximately 10 parts by weight of the chosen weight loss agent and approximately 200-250 parts by weight of lactose, which parts are disaggregated and blended. The mixture is then filled into hard gelatin capsules with each capsule containing a unit dose of the weight loss agent.

Also, by way of example, if the weight loss agent is administered in tablet form, the tablets can include approximately 10 parts by weight of the weight loss agent, 190 parts by weight lactose, 22 parts by weight maize starch, 10 parts by weight polyvinylpyrrolidone, and 3 parts by weight magnesium stearate. To manufacture the tablet by methods known in the art, the weight loss agent, lactose, and a portion of the maize starch are disaggregated, blended and the resulting mixture is granulated with a solution of the polyvinylpyrrolidone in ethanol. The dry granulate is blended with the magnesium stearate and remaining starch. The mixture is then compressed in a tabletting machine to make tablets each containing a unit dose or a part of a unit dose of the weight loss agent.

For enteric-coated tablets, the tablets made as above are coated in a convention manner using a solution of 20% cellulose acetate phthalate and 33% diethyl phthalate in ethanol:dichloromethane (1:1).

If the meal replacement formulation is administered in liquid form, preferably the amount of liquid can be from 1 fluid ounce to about 24 fluid ounces and have a caloric value of approximately 50 kcal to 1000 kcal. Most preferably, if administered in liquid form, the meal replacement formulation can be from about 6 fluid ounces to 12 fluid ounces and have a caloric value of approximately 150 kcal to 400 kcal.

If the meal replacement formulation is administered in the form of a bar or biscuit, preferably the bar can range from approximately 20 g to 150 g and have a caloric value of from 50 kcal to 1000 kcal. More preferably, the bar or biscuit can range from approximately 30 g to 60 g and have a caloric value of approximately 150 kcal to 400 kcal. If the meal replacement formulation is administered in powder form for dissolution in a liquid medium, e.g., water, milk, juice, etc., preferably the amount of powder can be approximately 25 g to 150 g/serving and can have a caloric value from approximately 50 kcal to 1000 kcal. Most preferably, the powder can be approximately 30 g to 60 g/serving and have a caloric value of approximately 150 kcal to 400 kcal.

Table 1 provides additional meal replacement formulations including available forms (bar, liquid, powder, pudding) and dosages of each suitable formulation for use in the present method.

TABLE 1

| Brand Name | Bar | Liquid | Powder | Pudding |
|---|---|---|---|---|
| Glucerna | 1.41 oz (40 g) and | 8 Fluid Ounces | 14-oz (397-g) and | — |
| | 1.34 oz (38 g) and | | ½ cup (57 g) | |
| | 2.04 oz (58 g) | | | |
| Glucerna SR | 1.41 oz (40 g) and | 8 Fluid Ounces | 14-oz (397-g) and | — |
| | 1.34 oz (38 g) and | | ½ cup (57 g) | |
| | 2.04 oz (58 g) | | | |
| Glucerna Weight | — | 11 Fluid Ounces | 14-oz (397-g) and | — |
| Loss Shake | | | ½ cup (57 g) | |
| Glucerna Select | — | 8 Fluid Ounces | — | — |
| Glucerna Snack | 1.41 oz (40 g) and | — | — | — |
| Bars | 1.34 oz (38 g) | | | |
| Glucerna Meal | 2.04 oz (58 g) | — | — | — |
| Bar | | | | |
| Glucerna Shake | — | 8 Fluid Ounces | — | — |
| Consumer | | | | |
| Ensure | 1.41 oz (40 g) and | 8 Fluid ounces | 14-oz (397-g) and | 4- oz cup |
| | 1.34 oz (38 g) and | | ½ cup (57 g) | |
| | 2.04 oz (58 g) | | | |
| Ensure Healthy | — | 8 Fluid ounces | | — |
| Mom Shake | | | | |
| Ensure Healthy | 0.70-oz (20 g) | — | — | — |
| Mom Bar | | | | |
| Ensure Fiber with | — | 8 Fluid ounces | 14-oz (397-g) and | — |
| FOS | | | ½ cup (57 g) | |
| Ensure High | — | 8 Fluid ounces | 14-oz (397-g) and | — |
| Calcium | | | ½ cup (5 7 g) | |
| Ensure High | — | 8 Fluid ounces | 14-oz (397-g) and | — |
| Protein | | | ½ cup (57 g) | |
| Ensure Plus | | 8 Fluid ounces | 14-oz (397-g) and | — |
| | | | ½ cup (57 g) | |
| Ensure Plus HN | — | 8 Fluid ounces | — | — |
| Prosure | — | 8 Fluid ounces | 14-oz (397-g) and | — |
| | | | ½ cup (57 g) | |
| Prosure with Fiber | — | 8 Fluid ounces | 14-oz (397-g) and | — |
| | | | ½ cup (57 g) | |
| Prosure with FOS | — | 8 Fluid ounces | 14-oz (397-g) and | — |
| | | | ½ cup (57 g) | |
| Zone Perfect | — | — | 2 scoops (59 g) | — |
| Shake Mix | | | | |
| Zone Perfect | — | 8 Fluid ounces | — | — |
| Shakes | | | | |
| Zone Perfect | 50 g | — | — | — |
| Nutrition Bar | | | | |
| Myoplex Deluxe | | — | 1 pack (96 g) | — |
| Myoplex Deluxe | 90 g | — | — | — |
| Bar | | | | |
| Myoplex Storm | 80 g | — | — | — |
| Bar | | | | |
| Myoplex Original | — | | 76 g | — |
| Powder MRP | | | | |
| Myoplex Original | — | 330 ml | — | — |
| Ready to Drink | | | | |
| Myoplex Lite | 56 g | — | — | — |
| Powder MRP | | | | |
| Myoplex Lite | — | 330 ml | — | — |
| Ready to Drink | | | | |
| Myoplex Lite Bar | 56 g | — | — | — |
| Myoplex Carb | — | 330 ml | — | — |
| Sense Ready to | | | | |
| Drink | | | | |
| Myoplex Carb | 76 g | — | — | — |
| Sense Powder | | | | |
| Myoplex Carb | — | 330 ml | — | — |
| Sense Protein | | | | |
| Drink | | | | |
| Myoplex Carb | 56 g | — | — | — |
| Sense Bar | | | | |
| Body for Life | — | 11 Fluid Ounces | — | — |
| Drink | | | | |
| Body for Life | — | 50 g | — | — |
| Powder | | | | |
| Optifast | | | | — |
| Modifast | | | | — |
| Boost Drink | — | 8 Fluid Ounces | — | — |
| Boost Benefiber | — | 8 Fluid Ounces | — | — |
| with FOS | | | | |

The meal replacement formulations listed above are particularly advantageous in that all provide a highly nutritious, palatable, and satisfying source of vitamins and minerals as well as carbohydrates and proteins and are, most importantly, low in fit and relatively low in calories. Advantageously, the meal replacement formulation and the weight loss agent forming the system of the invention can be sold as a single unit or kit with premeasured doses of each to be used when taken in conjunction with each other. The kit can include enough meal replacement formulations for one week, two weeks, one month, etc., and an equal number of prescribed individual doses of the weight loss agent, e.g., sibutramine, the specific dosage of the weight loss agent being matched or tailored to the contents of the meal replacement formulation and taking into account the particular needs of the individual utilizing the system for weight loss or weight loss maintenance. Alternatively, the weight loss agent, when packaged with multiple servings of the meal replacement formulation, can be divided into specific dosages corresponding to the prescribed frequency of administration, i.e., if the weight loss agent is to be taken more or less frequently than is the meal replacement formulation. For example, if the weight loss or weight loss maintenance regimen for an individual calls for the use of 20 mg/day of a weight loss agent together with replacement of two meals a day with a meal replacement formulation, then the weight loss agent can be prepackaged with the meal replacement formulation servings either in 20 mg doses (for once-a-day administration) or in 10 mg doses (so that a dose of weight loss agent can be taken together with each serving of a meal replacement formulation).

In another embodiment, the kit can include a number of meal replacement formulations and a combination of more than one type of weight loss agent, in particular, a first weight loss agent, for example, sibutramine, that is chemically distinct from a second weight loss agent, for example, rimonabant. In this embodiment, the first and second weight loss agents can be administered in an alternating cycle with each other wherein the first weight loss agent is administered with the meal replacement formulation for a set number of consecutive days, weeks, months, etc., then immediately following this set period, the second weight loss agent is administered with the meal replacement formulation preferably, but not necessarily for the same set number of days, weeks, months, etc., as the first weight loss agent. This alternating cycle can persist for the duration of the weight loss program. For example, according to this embodiment, in addition to the daily administration of the meal replacement formulation, sibutramine can be taken in an alternating cycle with rimonabant, wherein the sibutramine is taken with the meal replacement formulation for a first 7-day period, then the rimonabant is taken with the meal replacement formulation for a second 7-day period immediately following the first 7-day period This cycle can continue for the duration of the program. In another, embodiment, this administration can include a 14-day cycle wherein the sibutramine is taken with the meal replacement formulation for a first 14-day period, then the rimonabant is taken with the meal replacement formulation for a second 14-day period immediately following the first 14-day period. This cycle can continue for the duration of the program. In yet another embodiment, the administration can include an alternating 28-day cycle wherein the sibutramine is administered with the meal replacement formulation for a first 28-day period and the rimonabant is administered with the meal replacement formulation for a second 28-day period immediately following the first 28-day period. This alternating cycle, with cycles of any duration and either the same or different duration, can continue for the duration of the weight loss program.

In another particular embodiment, the meal replacement formulation and the weight loss agents can be provided in a 28-day kit including 14 doses/servings of sibutramine and meal replacement formulations and 14 doses/servings of rimonabant and meal replacement formulations.

In one embodiment of a kit that includes a combination of separate doses of sibutramine and rimonabant, the dose of sibutramine is between about 5 mg to about 30 mg of sibutramine hydrochloride monohydrate or the mole equivalent amount of a sibutramine compound; and between about 2 mg to about 20 mg of rimonabant hydrochloride or the mole equivalent amount of a rimonabant compound; and one or more pharmaceutically suitable carriers or excipients.

In another preferred embodiment, the dose of sibutramine is between about 5 mg to about 19 mg of sibutramine hydrochloride monohydrate or the mole equivalent amount of a sibutramine compound; and the dose of rimonabant is between about 3 mg to about 20 mg of rimonabant hydrochloride or the mole equivalent amount of a rimonabant compound; and one or more pharmaceutically suitable carriers or excipients.

In another preferred embodiment, the dose of sibutramine is between about 61 mg to about 18 mg of sibutramine hydrochloride monohydrate or the mole equivalent amount of a sibutramine compound; and the dose of rimonabant is between about 4 mg to about 20 mg of rimonabant hydrochloride or the mole equivalent amount of a rimonabant compound; and one or more pharmaceutically suitable carriers or excipients.

In another preferred embodiment, the dose of sibutramine is between about 7 mg to about 17 mg of sibutramine hydrochloride monohydrate or the mole equivalent amount of a sibutramine compound; and the dose of rimonabant is between about 5 mg to about 20 mg of rimonabant hydrochloride or the mole equivalent amount of a rimonabant compound; and one or more pharmaceutically suitable carriers or excipients.

In another preferred embodiment, the dose of sibutramine is between about 8 mg to about 16 mg of sibutramine hydrochloride monohydrate or the mole equivalent amount of a sibutramine compound; and the dose of rimonabant is between about 6 mg to about 20 mg of rimonabant hydrochloride or the mole equivalent amount of a rimonabant compound; and one or more pharmaceutically suitable carriers or excipients.

In another preferred embodiment, the dose of sibutramine is between about 9 mg to about 15 mg of sibutramine hydrochloride monohydrate or the mole equivalent amount of a sibutramine compound; and the dose of rimonabant is between about 7 mg to about 20 mg of rimonabant hydrochloride or the mole equivalent amount of a rimonabant compound; and one or more pharmaceutically suitable carriers or excipients.

In another preferred embodiment, the dose of sibutramine is between about 10 mg to about 15 mg of sibutramine hydrochloride monohydrate or the mole equivalent amount of a sibutramine compound; and the dose of rimonabant is between about 10 mg to about 20 mg of rimonabant hydrochloride or the mole equivalent amount of a rimonabant compound; and one or more pharmaceutically suitable carriers or excipients.

In another preferred embodiment, the dose of sibutramine is about 10 mg of sibutramine hydrochloride monohydrate or the mole equivalent amount of a sibutramine compound; and the dose of rimonabant is about 5 mg of rimonabant hydrochloride or the mole equivalent amount of a rimonabant compound; and one or more pharmaceutically suitable carriers or excipients.

In another preferred embodiment, the dose of sibutramine is about 15 mg of sibutramine hydrochloride monohydrate or the mole equivalent amount of a sibutramine compound; and the dose of rimonabant is about 5 mg of rimonabant hydrochloride or the mole equivalent amount of a rimonabant compound; and one or more pharmaceutically suitable carriers or excipients.

In another preferred embodiment, the dose of sibutramine is about 10 mg of sibutramine hydrochloride monohydrate or the mole equivalent amount of a sibutramine compound; and the dose of rimonabant is about 10 mg of rimonabant hydrochloride or the mole equivalent amount of a rimonabant compound; and one or more pharmaceutically suitable carriers or excipients.

In another preferred embodiment, the dose of sibutramine is about 10 mg of sibutramine hydrochloride monohydrate or the mole equivalent amount of a sibutramine compound; and the dose of rimonabant is about 15 mg of rimonabant hydrochloride or the mole equivalent amount of a rimonabant compound; and one or more pharmaceutically suitable carriers or excipients.

In another preferred embodiment, the dose of sibutramine is about 15 mg of sibutramine hydrochloride monohydrate or the mole equivalent amount of a sibutramine compound; and the dose of rimonabant is about 10 mg of rimonabant hydrochloride or the mole equivalent amount of a rimonabant compound; and one or more pharmaceutically suitable carriers or excipients.

In another preferred embodiment, the dose of sibutramine is about 15 mg of sibutramine hydrochloride monohydrate or the mole equivalent amount of a sibutramine compound; and the dose of rimonabant is about is mg of rimonabant hydrochloride or the mole equivalent amount of a rimonabant compound; and one or more pharmaceutically suitable carriers or excipients.

In another preferred embodiment, the dose of sibutramine is about 15 mg of sibutramine hydrochloride monohydrate or the mole equivalent amount of a sibutramine compound; and the dose of rimonabant is about 20 mg of rimonabant hydrochloride or the mole equivalent amount of a rimonabant compound; and one or more pharmaceutically suitable carriers or excipients.

According to the present invention, a weight loss agent is taken “in conjunction with” a meat replacement formulation, which means that the two components are administered to or taken by the individual seeking to lose weight at essentially the same time, or the components are both administered or consumed within a period of time such that the effects of both components are exhibited in the individual. Preferably, the meal replacement formulation component is administered at the same time as the weight loss agent component. Alternatively, the meal replacement formulation is administered within 5, 10, 15, 20, 30, 45, or 60 minutes of administering the weight loss agent, or within 2, 3, 4, 5, 6, 7, 8, 12, 16, 18, 24 hours of administering the weight loss agent, or at any interval in between. For example, the weight loss agent can be taken first thing in the morning and the meal replacement formulation can be taken later in the day, e.g., lunch or dinner, or as a snack. For simultaneous administrations, it is especially preferred that the weight loss agent and the meal replacement formulation can be co-formulated, that is, can be mixed together or co-dispersed in a common medium. This advantageously permits both components of the system to be ingested by the individual seeking weight loss or weight loss maintenance in a single administration, which increases compliance with the method of the invention and ensures success of the weight loss regimen.

The components of the weight control system of the invention are advantageously packaged together for convenient co-administration, and therefore in another embodiment, a kit is provided including appropriate dose(s) of the weight loss agent, serving(s) of a meal replacement formulation, and suitable instructions for the effective co-administration of the meal replacement formulation and weight loss agent. According to this embodiment, the dosages can be customized to the weight loss needs of the particular individual. For example, the kit can include the meal replacement formulation already premixed with the weight loss agent (co-formulation), or the weight loss agent can be packaged separately in the kit in individual doses to be taken in conjunction with the meal replacement formulation or admixed by the individual to the meal replacement formulation prior to ingestion.

It is envisioned that the method of the present invention can be performed in conjunction with a lifestyle/behavior modification program. For the purposes of the present invention, a “lifestyle modification program” is any instruction, activity, or information intended to induce individuals to make or continue changes in their behavior that increase the likelihood of their losing weight or maintaining a reduced weight. The two distinct mechanisms of action i.e., effect of the weight loss agent combined with the meal replacement, can be enhanced by applying a behavior modification program or lifestyle modification program. Behavior modification programs or lifestyle modification programs that can suitably be employed to enhance the effectiveness of the present invention include, without limitation, means for charting, recording or monitoring an individual's weight or body mass index (BMI), especially over time; instructional materials for following a particular diet or exercise regimen, or both; means for tracking, recording or monitoring food intake, especially over time; means for scheduling meetings to review progress or to review health status (i.e., “check-ups”); classes, lectures or demonstrations for learning or reinforcing learned lifestyle modifications; group support sessions; psychotherapy sessions; personal trainer or diet coach sessions; and the like. Instructional lifestyle modification programs that are not conducted in person by a professional administrator such as a doctor, nurse, dietician, trainer, monitor, pharmacist, leader, group leader, coach, teacher, etc. can take any tangible form such as sound recordings, video recordings, broadcasts through any mass medium, printed matter, models, pictures, charts, alarms, notices, pedometer, meal plans, a diary to record food intake and behavior modification tools to assist in changing eating behavior or any combination of the same.

Therefore, in another embodiment of the present invention, a kit according to the invention can include a lifestyle modification program, which provides educational instruction to an individual on any aspect of modifying and maintaining a lifestyle that is conducive to losing weight and/or maintaining a desired body weight, Particularly mentioned features of such an embodiment are kits made to include means for monitoring food intake, such as a food diary, means for keeping track of weight loss or gain, such as a weight diary or calendar, means for estimating or measuring caloric intake, such as a calorie counter, means for adopting a beneficial diet, such as menus or recipes for healthful meals, and like programs that can be conveniently packaged with the weight loss system described herein.

In a particularly preferred embodiment, the weight loss agent of the present invention is sibutramine, having the structure as shown in Formula I, including pharmaceutically acceptable salts thereof and enantiomers, in which R₁ and R₂ are independently H or methyl.

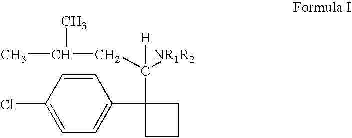

As used herein, the term “pharmaceutically acceptable salt” refers to a salt prepared from a pharmaceutically acceptable non-toxic inorganic or organic acid, Inorganic acids include, but are not limited to, hydrochloride, hydrobromide, sulfates, methanesulfonates (mesylate), nitrates, maleates, acetates, citrates, fumarates, tartrates, e.g., (+)-tartrates, (−)-tartrates or mixtures thereof including racemic mixtures, succinates, benzoates and salts with amino acids such as glutamic acid, hydrobromic, hydroiodic, nitric, sulfuric, and phosphoric. Organic acids include, but are not limited to, aliphatic, aromatic, carboxylic, and sulfonic organic acids including, but not limited to, formic, acetic, propionic, succinic benzoic camphorsulfonic, citric, fumaric, gluconic, isethionic, lactic, malic, mucic, tartaric, para-toluenesulfonic, glycolic, glucuronic, maleic, faroic, glutamic, benzoic, anthranilic, salicyclic, phenylacetic, mandelic, embonic (pamoic), methanesulfonic, ethanesulfonic, pantothenic, benzenesulfonic, stearic sulfanilic, alginic, and galacturonic acid as well as salts with acidic amino acids such as aspartic and glutamic acids. Also included in the pharmaceutically acceptable salts of sibutramine is a sibutramine methanesulfonate hemihydrate as described in U.S. Publication No. US 2004/0068018 A1, Particularly preferred acid salts are those of hydrobromic, hydrochloric, phosphoric, and sulfuric acids, and most particularly preferred is hydrochloric acid.

In a particularly preferred embodiment, the sibutramine compound of Formula I is N,N-dimethyl-1-[1-(4-chlorophenyl)cyclobutyl]-3-methylbutylamine or a salt thereof, for example the hydrochloride salt. A preferred form of this hydrochloride salt is its monohydrate.

In another preferred embodiment, the sibutramine compounds of Formula I suitable for use in the present method include N-{1-[-(4-chlorophenyl)cyclobutyl]-3-methylbutyl}-N-methylamine and 1-[1-(4-chlorophenyl)cyclobutyl]-3-methylbutylamine including clathrates, racemates, individual enantiomers, and mixtures thereof.

The most preferred sibutramine compounds of Formula I are sibutramine hydrochloride, sibutramine mesylate, sibutramine hydrochloride monohydrate, sibutramine mesylate hemihydrate, and sibutramine methanesulfonate hemihydrate.

It can be appreciated by those skilled in the art that compounds of Formula I contain a chiral center. When a compound of Formula I contains a single chiral center it can exist in two enantiomeric forms. The present invention includes the use of the individual enantiomers and mixtures of the enantiomers, which can be resolved by methods known in the alt.

The individual enantiomers of sibutramine can be prepared by enantioselective synthesis from optically active precursors, or by resolving the racemic compound, Enantiomers of secondary amines of Formula I can also be prepared by preparing the racemate of the corresponding primary amine, resolving the latter into the individual enantiomers, and then converting the optically pure primary amine enantiomer into the required secondary amine by methods known in the art.

Specific examples of sibutramine enantiomers of Formula I suitable for use in the present method include, but are not limited to:
(+)-N-[1-[(4-chlorophenyl)cyclobutyl]-3-methylbutyl}-N-methylamine;
(−)-N-{1-[1-(4-chlorophenyl)cyclobutyl-3-methylbutyl}-N-methylamine;
(+)-1-[1-(4-chlorophenyl)cyclobutyl]-3-methylbutylamine;
(−)-1-[1-(4-chlorophenyl)cyclobutyl]-3-methylbutylamine;
(+)-N-{1-[1-(4-chlorophenyl)cyclobutyl]-3-methylbutyl}-N—N-dimethyl amine;
(−)-N-{-[1-(4-chlorophenyl)cyclobutyl]-3-methylbutyl}-N—N-dimethylamine.

The hydrochloride salts are preferred in each case above, but the free bases and other pharmaceutically acceptable salts are also suitable for use in the present method. It is also contemplated by the present invention that other pharmaceutical salts, solvates, and clathrates of racemic and optically pure sibutramine metabolites are used in the methods and compositions of the present invention.

In another embodiment, the pharmaceutically acceptable salts of sibutramine can exist as hydrates. The degree of hydration can depend on the salt. The preferred hydrates are sibutramine hydrochloride monohydrate and sibutramine mesylate hemihydrate. The preparation of sibutramine hydrochloride and its monohydrate is described in U.S. Pat. No. 4,929,629 and the preparation of sibutramine mesylate and it hemihydrate is described in U.S. Pat. Public. No. US 2004/0068018, both of which are incorporated herein by reference.

In another embodiment, the compounds of Formula I are racemic and optically pure sibutramine metabolites. Examples of racemic and optically pure sibutramine metabolites include, but are not limited to, (+)-desmethylsibutramine, (−)-desmethylsibutramine, (+)-didesmethylsibutramine, and (−)-didesmethylsibutramine.

The amount or dosage of sibutramine to be administered with the meal replacement formulation of the method of the present invention can depend on a number of factors including the age of the individual, the severity of the condition, and the past medical history or current medical condition of the patient and always lies within the sound discretion of the administering physician.

It is generally envisioned that the dose of sibutramine, whether taken in conjunction with the meal replacement formulation or co-formulated with the meal replacement formulation, can be in the range of 0.1 to 100 mg/day, preferably 1 to 50 mg/day, more preferably 5 to 30 mg/day and most preferably 10-20 mg/day.

In another aspect, the dose of sibutramine according to the present method can be 5 mg/day, 10 mg/day, 15 mg/day, 20 mg/day, 25 mg/day, or 30 mg/day. Most preferably, according to this aspect of the invention, the most preferred dose of sibutramine can be 10 mg, 12 mg or 15 mg per day.

In another aspect of the present invention, the dose of sibutramine to be administered with the meal replacement formulation can be 1 mg/day, 2 mg/day, 3 mg/day, 4 mg/day, 5 mg/day, 6 mg/day, 7 mg/day, 8 mg/day, 9 mg/day, 10 mg/day, 11 mg/day, 12 mg/day, 13 mg/day, 14 mg/day, 15 mg/day, 16 mg/day, 17 mg/day, 18 mg/day, 19 mg/day, 20 mg/day, or 30 mg/day. In a most preferred aspect of this embodiment, the preferred dosage of sibutramine is 10 mg/day.

In another aspect of the present invention, the daily dosage in mg/day of sibutramine to be administered with the meal replacement formulation can be 1.0 mg/day, 1.25 mg/day, 1.50 mg/day, 1.75 mg/day, 2.0 mg/day, 2.25 mg/day, 2.50 mg/day, 2.75 mg/day, 3.0 mg/day, 3.25 mg/day, 3.50 mg/day, 3.75 mg/day, 4.0 mg/day, 4.25 mg/day, 4.50 mg/day, 4.75 mg/day, 5.0 mg/day, 5.25 mg/day, 5.50 mg/day, 575 mg/day, 6.0 mg/day, 6.25 mg/day, 6.50 mg/day, 6.75 mg/day, 7.0 mg/day, 7.25 mg/day, 7.50 mg/day, 7.75 mg/day, 8.0 mg/day, 8.25 mg/day, 850 mg/day, 8.75 mg/day, 9.0 mg/day, 9.25 mg/day, 9.50 mg/day, 9.75 mg/day, 10.0 mg/day, 10.25 mg/day, 10.50 mg/day, 10.75 mg/day, 11.0 mg/day, 11.25 mg/day, 11.50 mg/day, 11.75 mg/day, 12.0 mg/day, 12.25 mg/day, 12.50 mg/day, 12.75 mg/day, 13.0 mg/day, 13.25 mg/day, 13.50 mg/day, 13.75 mg/day, 14.0 mg/day, 14.25 mg/day, 14.50 mg/day, 14.75 mg/day, 15.0 mg/day, 15.25 mg/day, 15.50 mg/day, 15.75 mg/day, 16.0 mg/day, 16.25 mg/day, 16.50 mg/day, 16.75 mg/day, 17.0 mg/day, 17.25 mg/day, 17.50 mg/day, 17.75 mg/day, 18.0 mg/day, 18.25 mg/day, 18.50 mg/day, 18.75 mg/day, 19.0 mg/day, 19.25 mg/day, 19.50 mg/day, 19.75 mg/day, 20.0 mg/day, 30.0 mg/day.

In a preferred embodiment of the method of the present invention, when the meal replacement formulation is administered in a liquid formulation either co-formulated with the sibutramine or administered as a separate formulation than the sibutramine, the meal replacement formulation can be from about 1 fluid ounce to about 24 fluid ounces and have a caloric value of approximately 50 kcal to 1000 kcal, and the dose of sibutramine can be from about 0.1 to 10 mg, preferably about 5 to 50 mg, more preferably about 10 to 30 mg and most preferably the dose of sibutramine can be 100 mg. In a most preferred embodiment, the meal replacement formulation can be from about 6 fluid ounces to 12 fluid ounces and have a caloric value of approximately 150 kcal to 400 kcal., and the dosage of sibutramine can be from about 0.1 to 100 mg, preferably about 5 to 5 mg, more preferably about 10 to 30 mg and most preferably the dose of sibutramine can be about 10 mg.

In another preferred embodiment of the method of the present invention, if the meal replacement formulation is administered in the form of a bar or biscuit and either co-formulated with the sibutramine or administered in a separate dose from the sibutramine, preferably the bar or biscuit can range from about 20 to 150 g and have a caloric value of from 50 kcal to 1000 kcal., and the dose of sibutramine can be from about 0.1 to 100 mg, preferably about 5 to 50 mg, more preferably about 10 to 30 mg and most preferably the dose of sibutramine can be 10 mg. In a more preferred embodiment, the bar or biscuit can range from approximately 30 g to 60 g and have a caloric value of approximately 50 kcal to 400 kcal., and the dose of sibutramine can be from about 0.1 to 100 mg preferably about 5 to 50 mg, more preferably about 10 to 30 mg and most preferably the dose of sibutramine can be 10 mg.

In another preferred embodiment, if the meal replacement formulation is administered in powder form for dissolution in a liquid medium and either co-formulated with the sibutramine or administered in a separate dose from the sibutramine, preferably the amount of powder can be from about 25 g to 150 g/serving and have a caloric value from about 50 kcal to 1000 kcal., and the dose of sibutramine can be from about 0.1 to 100 mg, preferably about 5 to 50 mg, more preferably about 10 to 30 mg, and most preferably the dose of sibutramine can be 10 mg. In a most preferred embodiment, the powder can be about 30 g to 60 g/serving and have a caloric value of about 150 kcal to 400 kcal, and the dose of sibutramine can be from about 0.1 to 100 mg, preferably about 5 to 50 mg, more preferably about 10 to 30 mg and most preferably the dose of sibutramine can be 10 mg.

In another embodiment of the method of the present invention, the weight loss agent that can be taken in conjunction with or co-formulated with the meal replacement formulation is orlistat (Formula II) or a pharmaceutically acceptable salt of orlistat. 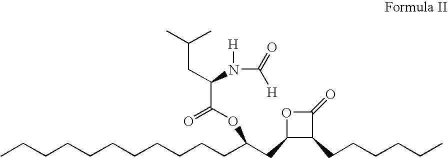
Orlistat is well known in the art as an inhibitor of lipase enzymes, which are responsible for breaking down ingested fat. (Borgstrom, B., Biochem Biophys Acta., 962(3): 308-316 (1988) The preferred form of orlistat is (2S,3S,5S)-5-[(S)-2-formamido-4-methylvaleryloxy]-2-hexyl-3-hydroxyhexadecanoic acid lactone. It is also known as N-formyl-L-leucine, ester with (3S,4S)-3-hexyl-4-[(2S)-2-hydroxytridecyl]-2-oxetanone, or (−)-tetrahydrolipstatin, and tetrahydrolipistatin.

The extraction and use of orlistat in the control or prevention of obesity and hyperlipidemia is described in U.S. Pat. No. 4,598,089. A process for the preparation of orlistat is described in U.S. Pat. No. 4,983,746, both of which are incorporated herein by reference.

The amount or dosage of orlistat to be administered with the meal replacement formulation of the present invention can depend on a number of factors including the age of the individual, the severity of the condition, and the past medical history or current medical condition of the patient and always lies within the sound discretion of the administering physician. In most cases, the dosage of orlistat to be administered according to the method of the present invention can be from about 50 mg to 1440 mg/day. More preferably the dosage of orlistat can be from about 120 mg to 720 mg/day. Most preferably, the dosage of orlistat according to the present method can be from about 120 mg to 360 mg. Even more preferably the dose of orlistat can be 120 mg/dose and administered in 1, 2, or 3 doses per day. If administered as an over the counter medicine, the dose of orlistat is preferably 60 mg/day.

In a preferred embodiment of the method of the present invention, when the meal replacement formulation is administered in a liquid formulation either co-formulated with the orlistat or administered is a separate formulation from the orlistat, the meal replacement formulation can be from about 1 fluid ounce to about 24 fluid ounces and have a caloric value of about 50 kcal to 1000 kcal., and the dose of orlistat can be from about 15 mg to 1440 mg, preferably about 120 to 720 mg, more preferably about 120 to 360 mg. In a most preferred embodiment, the meal replacement formulation can be from about 6 fluid ounces to 12 fluid ounces and have a caloric value of approximately 150 kcal to 400 kcal., and the dosage of orlistat can be from about 15 to 1440 mg., more preferably the dosage of orlistat can be from about 120 to 720 mg, and most preferably, the dosage of orlistat can be from about 120 to 360 mg.

In another preferred embodiment of the method of the present invention, if the meal replacement formulation is administered in the form of a solid bar or biscuit and either co-formulated with the orlistat of administered in a separate dose from the orlistat, preferably the bar or biscuit can range from about 20 g to 150 g and have a caloric value of from about 50 kcal to 1000 kcal., and the dose of orlistat can be from about 15 to 1440 mg, preferably from about 120 to 720 mg, and most preferably the dose of orlistat can be from about 120 to 360 mg. In a more preferred embodiment, the bar or biscuit can range from about 30 g to 60 g and have a caloric value of approximately 150 kcal to 400 kcal, and the dose of orlistat can be from about 15 to 1440 mg, preferably about 120 to 720 mg, and most preferably the dose of orlistat can be from about 120 to 360 mg.

If the meal replacement formulation is administered in powder form for dissolution in a liquid medium and either co-formulated with the orlistat or administered in a separate dose from the orlistat, preferably the amount of powder can be approximately 25 g to 150 g/serving and have a caloric value from about 50 kcal to 1000 kcal., and the dose of orlistat can be from about 15 to 1440 mg, preferably about 120 to 720 mg, and most preferably the dose of orlistat can be from about 120 to 360 mg. In a most preferred embodiment, the powder can be about 30 g to 60 g/serving and have a caloric value of about 150 kcal to 400 kcal., and the dose of orlistat can be from about 15 to 1440 mg, preferably about 120 to 720 mg, and most preferably the dose of orlistat can be from about 120 to 360 mg.

In another embodiment of the present invention, the weight loss agent that can be administered separately from the meal replacement formulation or co-formulated with the meal replacement formulation is rimonabant (Formula III) or a pharmaceutically acceptable salt of rimonabant. 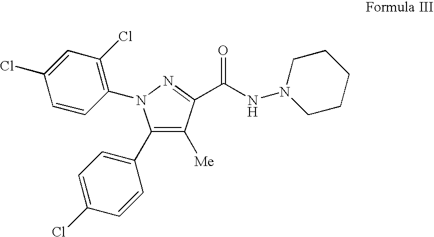
Rimonabant is also known as SR-147778 or by the chemical name 1-H-pyrazole-3-carboxamide, 5-(4-chlorophenyl)-1-(2-,4-dichlorophenyl)-4-methyl-N-1-piperidinyl. The preferred pharmaceutically acceptable salts of rimonabant are benzenesulfonate, hydrobromide, hydrochloride, citrate, ethanesulfonate, fumarate, gluconate, iodate, isothionate, maleate, methanesulfonate (mesylate), methylenebis-β-oxynaphthoate, nitrate, oxalate, pamoate, phosphate, salicylate, succinate, sulfate, tartrate, theophyllinacetate and p-toluenesulfonate. The preferred salts of rimonabant are the hydrochloride, mesylate and tartrate. The most preferred salt of rimonabant is rimonabant hydrochloride.

In a preferred embodiment, the dose of rimonabant, whether taken in conjunction with the meal replacement formulation or co-formulated with the meal replacement formulation, can be from about 2 to 20 mg/day, preferably about 5 to 15 mg/day, more preferably about 7 to 10 mg/day and most preferably 10 mg/day.

In another aspect, the dose of rimonabant according to the present method can be 2 mg/day, 5 mg/day, 10 mg/day, 15 mg/day, or 20 mg/day, depending on the weight loss needs of the individual. Most preferably, according to this aspect of the invention, the most preferred dose of rimonabant can be about 10 mg/day.

In another aspect of the present invention, the dose of rimonabant to be administered with the meal replacement formulation can be 1 mg/day, 2 mg/day, 3 mg/day, 4 mg/day, 5 mg/day, 6 mg/day, 7 mg/day, 8 mg/day, 9 mg/day, 10 mg/day, 11 mg/day, 12 mg/day, 13 mg/day, 14 mg/day, 15 mg/day, 16 mg/day, 17 mg/day, 18 mg/day, 19 mg/day, or 20 mg/day, depending on the weight loss needs of the individual. In a most preferred aspect of this embodiment, the preferred dosage of rimonabant is 10 mg/day.

In another aspect of the present invention, the daily dosage in mg/day of rimonabant to be administered with the meal replacement formulation can be 2.0 mg/day, 2.25 mg/day, 2.50 mg/day, 2.75 mg/day, 30 mg/day, 3.25 mg/day, 3.50 mg/day, 3.75 mg/day, 4.0 mg/day, 4.25 mg/day, 4.50 mg/day, 4.75 mg/day, 5.0 mg/day, 5.25 mg/day, 5.50 mg/day, 5.75 mg/day, 6.0 mg/day, 6.25 mg/day, 6.50 mg/day, 6.75 mg/day, 7.0 mg/day, 7.25 mg/day, 7.50 mg/day, 7.75 mg/day, 8.0 mg/day, 8.25 mg/day, 8.50 mg/day, 8.75 mg/day, 9.0 mg/day, 9.25 mg/day, 9.50 mg/day, 9.75 mg/day, 10.0 mg/day, 10.25 mg/day, 10.50 mg/day, 10.75 mg/day, 11.0 mg/day, 11.25 mg/day, 11.50 mg/day, 11.75 mg/day, 12.0 mg/day, 12.25 mg/day, 12.50 mg/day, 12.75 mg/day, 13.0 mg/day, 13.25 mg/day, 13.50 mg/day, 13.75 mg/day, 14.0 mg/day, 14.25 mg/day, 14.50 mg/day, 14.75 mg/day, 15.0 mg/day, 15.25 mg/day, 15.50 mg/day, 15.75 mg/day, 16.0 mg/day, 16.25 mg/day, 16.50 mg/day, 16.75 mg/day, 17.0 mg/day, 17.25 mg/day, 17.50 mg/day, 17.75 mg/day, 18.0 mg/day, 18.25 mg/day, 18.50 mg/day, 18.75 mg/day, 19.0 mg/day, 19.25 mg/day, 19.50 mg/day, 19.75 mg/day, or 20.0 mg/day.

In a preferred embodiment of the method of the present invention, when the meal replacement formulation is administered in a liquid formulation either co-formulated with the rimonabant or administered is a separate formulation from the rimonabant, the meal replacement formulation can be from about 1 fluid ounce to about 24 fluid ounces and have a caloric value of about 50 kcal to 1000 kcal., and the dose of rimonabant can be from about 2 to 20 mg, preferably about 5 to 15 mg, more preferably about 7 to 10 mg and most preferably the dose of rimonabant can be about 10 mg. In a most preferred embodiment, the meal replacement formulation can be from about 6 fluid ounces to 12 fluid ounces and have a caloric value of approximately 150 kcal to 400 kcal., and the dose of rimonabant can be from about 2 to 20 mg, preferably about 5 to 15 mg, more preferably about 7 to 10 mg and most preferably the dose of rimonabant can be about 10 mg/day.

In another preferred embodiment of the method of the present invention, if the meal replacement formulation is administered in the form of a bar or biscuit and either co-formulated with the rimonabant or administered in a separate dose from the rimonabant, preferably the bay or biscuit can range from about 20 g to 150 g and have a caloric value of from about 50 kcal to 1000 kcal., and the dose of rimonabant can be from about 2 to 20 mg, preferably about 5 to 15 mg, more preferably about 7 to 10 mg and most preferably the dose of rimonabant can be about 10 mg. In a more preferred embodiment, the bar or biscuit can range from about 30 g to 60 g and have a caloric value of about 150 kcal to 400 kcal., and the dose of rimonabant can be from about 2 to 20 mg, preferably about 5 to 15 mg, more preferably about 7 to 10 mg and most preferably the dose of rimonabant can be about 10 mg.

In another preferred embodiment, if the meal replacement formulation is administered in powder form for dissolution in a liquid medium and either co-formulated with the rimonabant or administered in a separate dose from the rimonabant, preferably the amount of powder can be from about 25 g to 150 g/serving and have a caloric value from about 50 kcal to 1000 kcal., and the dose of rimonabant can be from about 2 to 20 mg, preferably about 5 to 15 mg, more preferably about 7 to 10 mg and most preferably the dose of rimonabant can be about 10 mg.

In a most preferred embodiment, the powder can be from about 30 g to 60 g/serving and have a caloric value of about 150 kcal to 400 kcal., and the dose of rimonabant can be from about 2 to 20 mg, preferably about 5 to 15 mg, more preferably about 7 to 10 mg and most preferably the dose of rimonabant can be about 10 mg.

Other weight loss agents suitable for use in the method of the present invention include, but are not limited to, appetite suppressants, for example, Phentermine, Methamphetamine, Benzphetamine, Amphetamines, Phenylpropanolamine, Phendimetrazine, Diethylpropion, Mazindol, Ephedrine, Pseudoephedrine, lipase inhibitors, such as Cetilistat (Alizyme, UK), GT-389-255 (Genzyme), selective cannabinoid CB1 receptor antagonists such as Surinabant (Sanofi-Aventis), FEB2002 (Bayer), SLV319 (Solvay), CP945598 (Pfizer), selective 5-HT2C receptor agonists such as APD356 (Arena), Beta 3 adrenergic receptor agonists such as Solabegron (GSK), neuropeptide-Y (PYY) analogs such as YY3-36 (Nastech), thermogenesis mediators such as Metreleptin (Amgen), KB2115-Thyroid hormone agonist (EndoChem), incretin (GLP-1) and amylin analogs and DPPIV inhibitors such as Exenatide (Amylin), and Pramlintide (Amylini), hormone ciliary neurotrophic factor (CNTF) Depiclermin/Axokine (Regeneron), cholecystolkinin (CCK) agonists such as NLC 002 (Nobex), dopamine agonists such as Bromocriptine, ghrelin antagonists such as NOXB11 (Merck and Noxxon), melanocortin MC4 agonists such as NNC 70-619 (Novo Nordisk), selective serotonin reuptake inhibitors (SSRI) such as Fluoxetine, antihyperglycemic drugs such as Metformin, Insulin, and Glucagons, androgens such as Dehydropepiandrosterone, Etiocholandione, Testosterone.

Other weight loss agents suitable for use in the method of the present invention include NPY-1 antagonists, histamine H3 receptor antagonists, tyrosine phosphatase inhibitors, caffeine, hoodia (herb), and topirimate.

### EXAMPLES

It was demonstrated through comparative historical studies that improved weight loss in obese/overweight patients could be achieved by following a weight loss regimen that included co-administration of a meal replacement formulation and a weight loss agent, as compared to patients administered either a meal replacement formulation alone or a weight loss agent alone, even when either the meal replacement formulation alone or the weight loss agent alone was administered in conjunction with a lifestyle modification designed to enhance/encourage adherence to the weight loss regimen.

To conduct the studies, patients who were diagnosed as overweight or obese were chosen for clinical trials to assess weight loss as a function of prescribed meal replacements or a weight loss agent (sibutramine). Co-administration of both a weight loss agent and meal replacement was also tested.

All patients received lifestyle modification training of either a moderate (data Groups 1-9, 11, and 12) or intensive (data Group 10) character which included a patient's participation in record keeping, professional consultation, and exercise in addition to the dietary protocol. Patients placed on a “Moderate” lifestyle management program were given a diet sheet with instructions for a defined intake of daily calories ranging from 800-1600 kcal, or a 600 kcal deficit diet, with or without exercise and routine visits with the Primary Care Provider to reinforce diet and behavior modification, but relying primarily on patient self-monitoring. Patients placed on an “Intensive” lifestyle management program were given a diet sheet with a defined intake of daily calories ranging from 800-1600 kcal, or a 600 kcal deficit diet, with meals recorded in a food diary, and were provided with ongoing diet counseling and caloric adjustment according to weight loss “Intensive” patients were also given defined minimal weekly requirements for exercise and instructed to record their activities in an exercise diary that was then collected and reviewed by the Primary Care Provider. The Intensive program also included routine visits throughout the course of the study with a Dietitian and the Primary Care Provider in order to reinforce diet and behavior modification.

Patients in some groups (see Table 2, data Groups 2, 11, and 12) utilized meal replacement formulations. These individuals were given a diet sheet with a defined intake of daily calories ranging from 800-1600 kcal, or a 600 kcal deficit diet, with a meal replacement formulation taken once or twice daily instead of a food meal, and routine visits with a Primary Care Provider to reinforce diet and behavior modification, but relying primarily on patient self-monitoring.

The remainder of the patients (see Table 2, data Groups 3-11) also received daily doses of sibutramine at 10 mg/day (data Groups 3-7 and 11) or 15 mg/day (data Groups 8, 9, and 12). Individuals in data Groups 11 and 12 were given a diet sheet with a defined intake of daily calories ranging from 800-1600 kcal, or a 600 kcal deficit diet, with a meal replacement formulation (SlimFast®) taken once or twice daily instead of a food meal where applicable (see Table 2), and routine visits with a Primary Care Provider to reinforce diet and behavior modification, but relying primarily on patient self-monitoring. Patients in data Group 10 were placed on a 10 mg/day sibutramine and “Intensive” life style management regimen with no meal replacement.

In each of the studies, patients were monitored for weight loss over a 12- or 24-week period. The data groups ranged in size from 17 to 592 patients, and were placed on either a conventional diet with moderate lifestyle management program (data Group 1), or a diet including a meal replacement component including substitution of either one or two meals per day with a meal replacement formulation, as part of a moderate lifestyle management (data Group 2), or a conventional diet plus one daily dose of sibutramine (10 mg) with a moderate or intensive lifestyle management (data Groups 3-7 and 10), a conventional diet plus one daily dose of sibutramine (15 mg) with moderate lifestyle management (data Groups 8 and 9), and finally, a diet with one or two meal replacements (SlimFast®) plus one daily dose of sibutramine (10 mg) and moderate lifestyle management (data Group 11) or a diet with one or two meal replacements (SlimFast®) plus one daily dose of sibutramine (10 mg for 12 weeks followed by 15 mg for 12 weeks) (data Group 12). The compiled results of the studies are shown in Table 2.

Table 2 is a table presenting grouped data from historical studies showing the superior effects of using the system for weight loss in accordance with the invention when compared to regimens utilizing either a weight loss agent alone or a meal replacement formulation alone. All subjects received moderate or intensive lifestyle management training. Data Group 1 received this lifestyle management alone, data Group 2 received meal replacement therapy in addition, data Groups 3-10 received varying regimens of sibutramine therapy alone, data Groups 11 and 12 received the system of the present invention.

As used herein, the term “conventional diet” refers to a traditional reduced calorie diet (RCD) plan that utilizes a food regimen. The reduced calorie diet is calculated based on the patient's height, weight and activity level. Basically, the patient's average preprogram kcal intake per day are determined, then from the start of the weight loss program, approximately 500 kcal/day are reduced from the patients diet until the optimum kcal/day intake level is achieved. A meal plan is provided based on the recommended calorie level and patients are instructed on how to follow the plan.

As seen in Table 2, total weight loss varied according to the particular weight loss regimen, Data Group 1, which was put on a conventional diet including moderate lifestyle management observed a mean weight loss of 32 kg at week 12. Data Group 2, which included a meal replacement plus a moderate lifestyle management program and no sibutramine, experienced an average weight loss of 5.6 kg when the latest observation to week 12 was assessed Patients administered only the weight loss agent sibutramine with no meal replacement exhibited a range of weight loss from 3.7 kg (data Group 3=10 mg sibutramine/day+moderate lifestyle management) at a to 8.4 kg (data Group 10=10 mg sibutramine/day+intensive lifestyle management) when the latest observation to week 12 was assessed.

However, the difference in weight loss between the group that demonstrated the least overall loss in weight and the group that demonstrated the greatest overall loss in weight, was observed between a group that received sibutramine alone with no meal replacement (data Group 5) and a group that received sibutramine plus a meal replacement (data Group 12), respectively. As seen in Table 2, data Group 5, which was placed on a 10 mg/day sibutramine plus moderate lifestyle management with no meal replacement observed a mean weight loss of 3.7 kg at the latest observation to week 12 time point. In contrast, data Group 12, which received 10 mg/day sibutramine for the first 12 weeks followed immediately by 15 mg/day sibutramine for the next 12 weeks, plus moderate lifestyle management, plus meal replacement, observed an overall average weight loss of 9.0 kg.

In addition, the average weight loss of all groups receiving 10 mg/day sibutramine, moderate lifestyle management and no meal replacement for 12 weeks, i.e., data Groups 3-7, had an overall average weight loss of 4.6 kg, whereas the group that received 10 mg/day sibutramine plus moderate lifestyle management plus meal replacement (data Group 11) had an overall average weight loss of 8.3 kg, which represents an approximately 50% improvement over sibutramine alone. In addition, the higher 15 mg dose of sibutramine plus moderate lifestyle management with no meal replacement for 24 weeks (data Groups 8 and 9) only improved average weight loss by approximately 25% (compare average weight loss of data Groups 8 and 9, i.e., 6.0 kg with data Group 12, 9.0 kg).

Also, as seen in Table 2 there was a small weight loss difference over a 12 week period between subjects administered a meal replacement and moderate lifestyle management without sibutramine (data Group 2) (average weight loss 5.6 kg) and patients administered 10 mg sibutramine/day+moderate lifestyle management without a meal replacement (data Groups 3-7) (range of weight loss: 3.7 kg-6.2 kg).

However, as seen in Table 2 (data Groups 11 and 12), a profound weight loss of 83 and 9.0 kg was achieved in a 12- and 24-week periods, respectively, using either a system of 10 mg sibutramine/day+SlimFast® meal replacement formulation for 12 weeks (data Group 11), or the system as described above over a period of 24 weeks including administration of 10 mg/day sibutramine for the first 12 weeks followed by administration of 15 mg/day sibutramine for the next 12 weeks, co-administered with SlimFast® meal replacement formulation (data Group 12). Patients on the latter regimen experienced an average weight loss of 9-0 kg at the 24-week time point (data Group 12).

In addition, patients were monitored within each group for their compliance with their respective program for the frill 12 week or 24 week period, depending on the group. The basic assumption was that generally patients who attended the designated visits were also taking the weight loss agent and/or meal replacement formulation and following the prescribed lifestyle management regimen. It was assumed that continued attendance at these visits demonstrated a continued interest on the part of the patient in participation in the trial, which was indicative of that patient's “compliance” with the requirements of the regimen.

As seen in Table 2, the patient attendance rate in the combination sibutramine+meal replacement+moderate lifestyle management groups, i.e., those administered 10 mg/day sibutramine weight loss agent+SlimFast® meal replacement formulation+moderate lifestyle management (data Group 11) was 83% over the 12 week period, and the patient attendance in the 10 mg/day or 15 mg/day sibutramine weight loss agent+SlimFast®+moderate lifestyle management was 60% (data Group 12). In contrast, the average attendance for groups receiving sibutramine alone (10 g/day or 15 mg/day)+moderate or intensive lifestyle management over a 12 or 24 week period, as a whole, demonstrated a significantly lower average compliance rate, i.e., 40%, (data Groups 3-10, range=9%-79%).

Therefore, the results in Table 2 clearly demonstrate that not only does the weight loss system of the present invention, utilizing administration of a meal replacement component in conjunction with a weight loss agent, result in better weight loss than either the meal replacement component or the weight loss agent alone, the results also demonstrate that the system leads to comparatively high patient compliance.

TABLE 2

| | | | | | | | Mean | |
|---|---|---|---|---|---|---|---|---|
| | | | | | | | Weight | Attendence |
| | | Meal | Sibutramine | Clinical | Mean | Sample | Change | at 12 or 24 |
| Data | Lifestyle | Replacement | Dose | Data | Baseline | Size | (kg) | week visit |
| Group | Management | (MR) | (mg/day) | (weeks) | Weight (kg) | (LOCF*) | (LOCF*) | (%) |
| 1 | Moderate | NO | 0 | 12 | 86.8 | 238 | −3.2 | 81 |
| 2 | Moderate | YES | 0 | 12 | 87.4 | 248 | −5.6 | 84 |
| 3 | Moderate | NO | 10 | 12 | 98.2 | 81 | −3.9 | 63 |
| 4 | Moderate | NO | 10 | 12 | 92.2 | 75 | −6.2 | 19 |
| 5 | Moderate | NO | 10 | 12 | 88.4 | 151 | −3.7 | 9 |
| 6 | Moderate | NO | 10 | 12 | 91.9 | 17 | −5 | 41 |
| 7 | Moderate | NO | 10 | 12 | 94.9 | 179 | −4.2 | 45 |
| 8 | Moderate | NO | 15 | 24 | 96.7 | 55 | −6.4 | 33 |
| 9 | Moderate | NO | 15 | 24 | 94.4 | 186 | −5.6 | 34 |
| 10 | Intensive | NO | 10 | 12 | 102.6 | 592 | −8.4 | 79 |
| 11 | Moderate | YES | 10 | 12 | 97.9 | 141 | −8.3 | 83 |
| 12 | Moderate | YES | 10 (12 | 24 | 97.9 | 86 | −9 | 60 |
| | | | weeks)/15 | | | | | |
| | | | (12 weeks) | | | | | |

*LOCF: Last Observation Carried forward

Additional embodiments of the present invention can be apparent to those skilled in the art from considering the foregoing disclosure. All such additional embodiments are within the scope of the present invention as defined in the claims to follow.

What is claimed is:

1. A method for effecting weight loss or weight loss maintenance in an individual seeking to lose weight or maintain weight loss comprising co-administration to said individual of a) a weight loss agent, and b) a meal replacement formulation, wherein the co-administration to said individual of said weight loss agent (a) in combination with said meal replacement formulation (b), effects a greater weight loss in said individual than the administration of either agent (a) or (b) alone.

2. The method according to claim 1, wherein said weight loss agent and said meal replacement formulation are administered in a co-formulation.

3. The method according to claim 1, wherein said weight loss agent and said meal replacement formulation are administered in separate doses.

4. The method according to claim 1, wherein said weight loss agent is sibutramine or a pharmaceutically-acceptable salt of sibutramine.

5. The method according to claim 4, wherein said weight loss agent is an enantiomer of sibutramine selected from the group consisting of:
(+)-N-[1-[(4-chlorophenyl)cyclobutyl]-3-methylbutyl}-N-methylamine;
(−)-N-{1-[1-(4-chlorophenyl)cyclobutyl-3-methylbutyl}-N-methyl amine;
(+)-1-[1-(4-chlorophenyl)cyclobutyl]-3-methylbutylamine;
(−)-1-[1-(4-chlorophenyl)cyclobutyl]-3-methylbutylamine;
(+)-N-{1-[1-(4-chlorophenyl)cyclobutyl]-3-methylbutyl}-N—N-dimethylamine; and
(−)-N-{1-[1-(4-chlorophenyl)cyclobutyl]-3-methylbutyl}-N—N-dimethylamine.

6. The method according to claim 4, wherein said sibutramine is administered in a dose of 5 mg/day.

7. The method according to claim 4, wherein said dose of sibutramine is administered in a dose selected from the group consisting of 10 mg/day, 15 mg/day, 20 mg/day, 25 mg/day, and 30 mg/day.

8. The method according to claim 1 wherein the meal replacement formulation is selected from the group consisting of a liquid, a bar, and a powder, and the weight loss agent is sibutramine.

9. The method according to claim 8, wherein the dose of sibutramine is 5 mg/day.

10. The method according to claim 8, wherein the dose of sibutramine is selected from the group consisting of 10 mg/day, 15 mg/day, 20 mg/day, 25 mg/day and 30 mg/day.

11. The method according to claim 2, wherein said meal replacement formulation and said weight loss agent are co-formulated in a liquid.

12. The method according to claim 2, wherein said meal replacement formulation and said weight loss agent are co-formulated in a bar.

13. The method according to claim 2, wherein said meal replacement formulation and said weight loss agent are co-formulated in a powder.

14. A system for effecting improved weight loss or weight maintenance in an individual seeking to lose weight or maintain weight loss comprising (a) a weight loss agent and (b) a meal replacement formulation, said system having increased effectiveness for causing weight loss than either component used alone.

15. The system according to claim 14, wherein said weight loss agent and said meal replacement formulation are administered in a co-formulation.

16. The system according to claim 14, wherein said weight loss agent and said meal replacement formulation are administered in separate doses.

17. The system according to claim 14, wherein said weight loss agent is sibutramine or pharmaceutically-acceptable salt of sibutramine.

18. The system according to claim 17, wherein said sibutramine is N,N-dimethyl-1-[1-(4-chlorophenyl)cyclobutyl]-3-methylbutylamine or a pharmaceutically-acceptable salt thereof.

19. The system according to claim 18, wherein said salt of sibutramine is the hydrochloride salt.

20. The system according to claim 19, wherein said hydrochloride salt is its monohydrate salt.

21. The system according to claim 17, wherein said sibutramine is N-{1-[-(4-chlorophenyl)cyclobutyl]-3-methylbutyl}-N-methyl amine and 1-[1-(4-chlorophenyl)cyclobutyl]-3-methylbutylamine or pharmaceutically acceptable salts thereof.

22. The system according to claim 21, wherein said salt of sibutramine is the hydrochloride salt.

23. The system according to claim 22, wherein said hydrochloride salt is its monohydrate salt.

24. The system according to claim 17, wherein said sibutramine is selected from the group consisting of sibutramine hydrochloride, sibutramine mesylate, sibutramine hydrochloride monohydrate, sibutramine mesylate hemihydrate, and sibutramine methanesulfonate hemihydrate.

25. The system according to claim 17, wherein said weight loss agent is an enantiomer of sibutramine.

26. The system according to claim 25, wherein said enantiomer is selected from the group consisting of:
(+)-N-[1-[(4-chlorophenyl)cyclobutyl]-3-methylbutyl}-N-methylamine;
(−)-N-{1-[1-(4-chlorophenyl)cyclobutyl-3-methylbutyl}-N-methylamine;
(+)-1-[1-(4-chlorophenyl)cyclobutyl]-3-methylbutylamine;
(−)-1-[1-(4-chlorophenyl)cyclobutyl]-3-methylbutylamine;
(+)-N-{1-[1-(4-chlorophenyl)cyclobutyl]-3-methylbutyl}-N—N-dimethylamine; and
(−)-N-{1-[1-(4-chlorophenyl)cyclobutyl]-3-methylbutyl}-N—N-dimethylamine.

27. The system according to claim 17, wherein said sibutramine is administered in a dose of 0.1 mg/day to 100 mg/day.

28. The system according to claim 27, wherein said dose of sibutramine is 1 mg/day to 50 mg/day.

29. The system according to claim 27, wherein said dose of sibutramine is 5 mg/day.

30. The system according to claim 27, wherein said dose of sibutramine is selected from the group consisting of 10 mg/day, 15 mg/day, 20 mg/day, and 25 mg/day and 30 mg/day.

31. The system according to claim 14, wherein said meal replacement formulation is selected from the group comprising a liquid, a bar, and a solid.

32. A weight control regimen kit comprising (a) one or more weight loss agents and (b) one or more meal replacement formulations, the amounts of (a) and (b) being adapted for use in combination to promote more effective weight loss or weight loss maintenance than is achieved using either component (a) or (b) alone.

33. The kit according to claim 32, wherein said weight loss agent and said meal replacement formulation are administered in a co-formulation.

34. The kit according to claim 32, wherein said weight loss agent and said meal replacement formulation are administered in separate doses.

35. The kit according to claim 32, wherein said weight loss agent is sibutramine or pharmaceutically-acceptable salt of sibutramine.

36. The kit according to claim 35, wherein said sibutramine is N,N-dimethyl-1-[1-(4-chlorophenyl)cyclobutyl]-3-methylbutylamine or a pharmaceutically-acceptable salt thereof.

37. The kit according to claim 36, wherein said salt of sibutramine is the hydrochloride salt.

38. The kit according to claim 37, wherein said hydrochloride salt is its monohydrate salt.

39. The kit according to claim 35, wherein said sibutramine is N-{1-[-(4-chlorophenyl)cyclobutyl]-3-methylbutyl}-N-methylamine and 1-[1-(4-chlorophenyl)cyclobutyl]-3-methylbutylamine or pharmaceutically acceptable salts thereof.

40. The kit according to claim 39, wherein said salt of sibutramine is the hydrochloride salt.

41. The kit according to claim 40, wherein said hydrochloride salt is its monohydrate salt.

42. The kit according to claim 35, wherein said sibutramine is selected from the group consisting of sibutramine hydrochloride, sibutramine mesylate, sibutramine hydrochloride monohydrate, sibutramine mesylate hemihydrate, and sibutramine methanesulfonate hemihydrate.

43. The kit according to claim 35, wherein said weight loss agent is an enantiomer of sibutramine.

44. The kit according to claim 43, wherein said enantiomer is selected from the group consisting of:
(+)-N-[1-[(4-chlorophenyl)cyclobutyl]-3-methylbutyl}-N-methylamine;
(−)-N-{1-[1-(4-chlorophenyl)cyclobutyl-3-methylbutyl}-N-methylamine;
(+)-1-[1-(4-chlorophenyl)cyclobutyl]-3-methylbutylamine;
(−)-1-[1-(4-chlorophenyl)cyclobutyl]-3-methylbutylamine;
(+)-N-{1-[1-(4-chlorophenyl)cyclobutyl]-3-methylbutyl}-N—N-dimethylamine; and
(−)-N-{1-[1-(4-chlorophenyl)cyclobutyl]-3-methylbutyl}-N—N-dimethylamine.

45. The kit according to claim 35, wherein said sibutramine is administered in a dose of 0.1 mg/day to 100 mg/day.

46. The kit according to claim 45, wherein said dose of sibutramine is 1 mg/day to 50 mg/day.

47. The kit according to claim 45, wherein said dose of sibutramine is selected from the group consisting of 5 mg/day, 10 mg/day, 15 mg/day, 20 mg/day, 25 mg/day and 30 mg/day.

48. The kit according to claim 32, wherein said meal replacement formulation is selected from the group comprising a liquid, a bay, and a powder.

49. The kit according to claim 32, further comprising a lifestyle modification program.

50. The kit according to claim 32, wherein said weight loss agents are sibutramine and rimonabant.

51. The kit according to claim 50, wherein the dose of sibutramine is from 5 mg to 30 mg of sibutramine hydrochloride monohydrate or the mole equivalent amount of a sibutramine compound and the dose of rimonabant is from 2 mg to 20 mg of rimonabant hydrochloride or the mole equivalent amount of a rimonabant compound.

52. The kit according to claim 51, wherein the dose of sibutramine is from 5 mg to 19 mg of sibutramine hydrochloride monohydrate or the mole equivalent amount of a sibutramine compound and the dose of rimonabant is 3 mg to 20 mg of rimonabant hydrochloride or the mole equivalent amount of a rimonabant compound.

53. The kit according to claim 52, wherein the dose of sibutramine is from 6 mg to 18 mg of sibutramine hydrochloride monohydrate or the mole equivalent amount of a sibutramine compound and the dose of rimonabant is 4 mg to 20 mg of rimonabant hydrochloride or the mole equivalent amount of a rimonabant compound.

54. The kit according to claim 53, wherein the dose of sibutramine is from 7 mg to 17 mg of sibutramine hydrochloride monohydrate or the mole equivalent amount of a sibutramine compound and the dose of rimonabant is 5 mg to 20 mg of rimonabant hydrochloride or the mole equivalent amount of a rimonabant compound.

55. The kit according to claim 54, wherein the dose of sibutramine is from 8 mg to 16 mg of sibutramine hydrochloride monohydrate or the mole equivalent amount of a sibutramine compound and the dose of rimonabant is from 6 mg to 20 mg of rimonabant hydrochloride or the mole equivalent amount of a rimonabant compound.

56. The kit according to claim 55, wherein the dose of sibutramine is 9 mg to 15 mg of sibutramine hydrochloride monohydrate or the mole equivalent amount of a sibutramine compound and the dose of rimonabant is 7 mg to 20 mg of rimonabant hydrochloride or the mole equivalent amount of a rimonabant compound.

57. The kit according to claim 56, wherein the dose of sibutramine is 10 mg to 15 mg of sibutramine hydrochloride monohydrate or the mole equivalent amount of a sibutramine compound and the dose of rimonabant is 10 mg to 20 mg of rimonabant hydrochloride or the mole equivalent amount of a rimonabant compound.

58. The kit according to claim 51, wherein the dose of sibutramine is 10 mg of sibutramine hydrochloride monohydrate or the mole equivalent amount of a sibutramine compound and the dose of rimonabant is 5 mg of rimonabant hydrochloride or the mole equivalent amount of a rimonabant compound.

59. The kit according to claim 51, wherein the dose of sibutramine is 15 mg of sibutramine hydrochloride monohydrate or the mole equivalent amount of a sibutramine compound and the dose of rimonabant is 5 mg of rimonabant hydrochloride or the mole equivalent amount of a rimonabant compound.

60. The kit according to claim 51, wherein the dose of sibutramine is 10 mg of sibutramine hydrochloride monohydrate or the mole equivalent amount of a sibutramine compound and the dose of rimonabant is 10 mg of rimonabant hydrochloride or the mole equivalent amount of a rimonabant compound.

61. The kit according to claim 51, wherein the dose of sibutramine is 10 mg of sibutramine hydrochloride monohydrate or the mole equivalent amount of a sibutramine compound and the dose of rimonabant is 15 mg of rimonabant hydrochloride or the mole equivalent amount of a rimonabant compound.

62. The kit according to claim 51, wherein the dose of sibutramine is 15 mg of sibutramine hydrochloride monohydrate or the mole equivalent amount of a sibutramine compound and the dose of rimonabant is 10 mg of rimonabant hydrochloride or the mole equivalent amount of a rimonabant compound.

63. The kit according to claim 51, wherein the dose of sibutramine is 15 mg of sibutramine hydrochloride monohydrate or the mole equivalent amount of a sibutramine compound and the dose of rimonabant is 15 mg of rimonabant hydrochloride or the mole equivalent amount of a rimonabant compound.

64. The kit according to claim 51, wherein the dose of sibutramine is 15 mg of sibutramine hydrochloride monohydrate or the mole equivalent amount of a sibutramine compound and the dose of rimonabant is 20 mg of rimonabant hydrochloride or the mole equivalent amount of a rimonabant compound.

65. A kit of 7 doses/servings, 14 doses/servings, or 28 doses/servings comprising one or more weight loss agents; one or more meal replacement formulations; and instructions for the administration of said weight loss agents and said meal replacement formulations.

66. The package according to claim 65 wherein said weight loss agents are selected from the group consisting of sibutramine and rimonabant and the instructions are selected from the group consisting of administration of sibutramine for a 7 day period followed by administration of rimonabant for a 7 day period, administration of sibutramine for a 14 day period followed by administration of rimonabant for a 14 day period, and administration of sibutramine for a 28 day period followed by administration of rimonabant for a 28 day period.
